(19) United States
(12) Patent Application Publication
(10) Pub. No.: US 2024/0299209 A1
(43) Pub. Date: Sep. 12, 2024

(54) Vaginal Therapeutic Delivery Device

(71) Applicant: Herphoric, Inc., Hamilton, OH (US)

(72) Inventors: Paul Buss, Covington, KY (US); Donald Scott Copland, Brookville, IN (US); Mason John Makulinksi, Fairview Park, OH (US); Matt Merrell Dunham, Monument, CO (US); John Edward Cronin, Jericho, VT (US); Nancy Edwards Cronin, Jericho, VT (US); Michael J. Baker, Georgia, VT (US)

(73) Assignee: Herphoric, Inc., Hamilton, OH (US)

(21) Appl. No.: 18/668,490

(22) Filed: May 20, 2024

Related U.S. Application Data
(63) Continuation-in-part of application No. PCT/US24/12937, filed on Jan. 25, 2024.
(60) Provisional application No. 63/441,074, filed on Jan. 25, 2023.

(51) Int. Cl. A61F 6/06

(52) U.S. Cl. CPC A61F 6/06

(57) ABSTRACT

The present invention is a vaginal device platform comprising an insertable therapeutic delivery device for delivering one or more therapeutic agents, comprising any of pharmaceutical, medicinal, or holistic components, directly to the mucosa of the vaginal canal and a means of recovering the device. The delivery device platform may be used to dispense one or more therapeutic agents. In one embodiment, a static insert device is used, in another embodiment the insert device is used in an applicator to deliver a dosage amount from the applicator along with the insertion of the insert device.

### FIELD OF THE DISCLOSURE

The present disclosure is generally related to pharmaceutical delivery devices, particularly for delivering a pharmaceutical or other therapeutic agent to the mucosa of the vaginal canal.

### BACKGROUND OF THE INVENTION

Pharmaceuticals are generally administered via one of four routes, orally, intravenously, topically, and via mucosal membranes. The most effective and efficient method depends upon the type of pharmaceutical, its characteristics, and the location and nature of treatment. Typically, the most direct route, which gets the active ingredients in the pharmaceutical to the treatment site via the shortest distance through the body in the shortest amount of time reduces the likelihood and severity of side effects while additionally reducing the time required for the active ingredients to become effective while additionally minimizing the dose of the active ingredients.

Dosing is challenging for pharmaceuticals as they must consider the delivery route, physiological processes which may inhibit or delay the effectiveness of the active ingredients, and further limitations in dosing to prevent potentially severe side effects from the pharmaceutical. This often results in longer courses of multiple doses or larger doses which increase the likelihood of side effects. Topical and mucosal delivery routes also pose challenges comprising both delivery and contact time with patches being frequently used for topical delivery of pharmaceuticals and sprays being common for mucosal delivery routes. Unfortunately, sprays are not always comfortable or practical and dosing can be inconsistent and contact times limited.

Pharmaceuticals for vaginal disorders, such as menstrual pain, are typically treated via oral pharmaceuticals, as direct contact with the mucosal membranes of the vaginal canal is difficult to maintain due to fluid flow and drainage via the canal. Similarly, absorbent materials can become a breeding ground for viruses, bacteria, and other pathogens which can result in toxic shock syndrome and other harmful conditions.

The vaginal route of drug delivery has proven valuable because it allows for targeted delivery of drugs to the reproductive organs, enabling direct action at the site of action without extensive systemic exposure. Furthermore by minimizing systemic absorption, vaginal drug delivery can decrease the occurrence of systemic side effects often associated with other routes like oral or intravenous administration. This is advantageous when aiming for localized treatment without impacting the entire body. Still further vaginally administered drugs can bypass first-pass metabolism in the liver, leading to higher bioavailability compared to orally administered drugs. This means more of the drug reaches the target site intact, potentially requiring lower doses for therapeutic effects. The rich vascularization and permeability of the vaginal mucosa allow for rapid absorption of drugs, resulting in quicker onset of action compared to some other routes of administration. Vaginal delivery also offers a non-invasive, convenient, and discrete mode of administration. Controlled-release formulations can be developed for vaginal delivery, enabling sustained and controlled release of drugs over an extended period. This can lead to a consistent therapeutic effect without frequent dosing. Furthermore, by bypassing the gastrointestinal tract, vaginal delivery reduces the likelihood of gastrointestinal disturbances such as nausea, vomiting, or irritation that can occur with oral medications. Vaginal delivery is particularly relevant in conditions like gynecological infections, labor induction, fertility treatments, hormone replacement therapy, infectious disease prevention, and localized treatments for gynecologic cancers.

Therefore, a device is needed which is capable of delivering a pharmaceutical, or other therapeutic agents, directly to the mucosa of the vaginal canal and surrounding tissues which can facilitate an improved experience ranging from comfort, ease of use, rapid treatment and relief of symptoms, and a reduced occurrence of side effects. Ease of use and the ability to ensure a consistent dose, and features facilitating patient diagnosis are also desired.

### BRIEF DESCRIPTION OF THE DRAWINGS

The accompanying drawings, which are incorporated in and constitute a part of this specification, illustrate embodiments of the invention and, together with a general description of the invention given below, serve to explain the principles of the invention.

FIG. 1 illustrates a vaginal therapeutic delivery device, according to an embodiment of the invention.

FIGS. 2A and 2B are a side cross-sectional view and a side perspective view of a vaginal therapeutic delivery device according to an embodiment of the invention.

FIGS. 3A-3B are side cross-sectional views of alternative ends of a vaginal therapeutic delivery device according to an embodiment of the invention.

FIG. 4 is a side perspective view of a portion of a vaginal therapeutic delivery device with a therapeutic sheath, according to an embodiment of the invention.

FIG. 4A is a side perspective view of a portion of the therapeutic sheath of FIG. 4, according to an embodiment of the invention.

FIG. 4B is a front view of a portion of a therapeutic sheath, according to an embodiment of the invention.

FIG. 4C is another front view of a portion of an alternative therapeutic sheath, according to an embodiment of the invention.

FIG. 4D is a side view of an applicator for a vaginal therapeutic delivery device according to an embodiment of the invention.

FIG. 4E is a side view of a vaginal therapeutic delivery device according to an embodiment of the invention.

FIG. 5 is a schematic view of a vaginal therapeutic delivery device according to an embodiment of the invention.

FIG. 6 is a schematic view of a elements of a vaginal therapeutic delivery device according to an embodiment of the invention.

FIG. 7 is a schematic view of a elements of a vaginal therapeutic delivery device according to an embodiment of the invention.

FIG. 8 is a side view of a vaginal therapeutic delivery device according to an embodiment of the invention.

FIG. 9A is a perspective view of a vaginal therapeutic delivery device according to an embodiment of the invention.

FIG. 9B is a perspective view, in partial cross-section, of the vaginal therapeutic delivery device of FIG. 9A according to an embodiment of the invention.

FIG. 9C is a side view, in partial cross-section, of a vaginal therapeutic delivery device according to another embodiment of the invention.

FIG. 9D is a side view, in partial cross-section, of a vaginal therapeutic delivery device according to another embodiment of the invention.

FIG. 10A is a perspective view of a vaginal therapeutic delivery device according to another embodiment of the invention.

FIG. 10B is a perspective view, in partial cross-section, of the vaginal therapeutic delivery device of FIG. 10A according to an embodiment of the invention.

FIG. 11A is a perspective view of a vaginal therapeutic delivery device according to another embodiment of the invention.

FIG. 11B is a side view, in partial cross-section, of the vaginal therapeutic delivery device of FIG. 11A according to an embodiment of the invention.

FIGS. 11C and 11D are side views, in partial cross-section, of the vaginal therapeutic delivery device of FIG. 11A according to an embodiment of the invention shown in various deployed positions.

FIG. 11B is a perspective view, in partial cross-section, of the vaginal therapeutic delivery device of FIG. 11A according to an embodiment of the invention.

FIG. 12 is a side view, in partial cross-section, of a vaginal therapeutic delivery device according to another embodiment of the invention.

FIG. 13 is a side view, in partial cross-section, of a vaginal therapeutic delivery device according to another embodiment of the invention.

FIG. 14A is a side view, in partial cross-section, of a vaginal therapeutic delivery device according to another embodiment of the invention.

FIG. 14B is a side view, in partial cross-section, of the vaginal therapeutic delivery device of FIG. 14A in an inflated state.

FIG. 15A is a side view, in partial cross-section, of a vaginal therapeutic delivery device according to another embodiment of the invention.

FIG. 15B is a side view, in partial cross-section, of the vaginal therapeutic delivery device of FIG. 15A in an inflated state.

FIG. 16 is a side view, in partial cross-section, of a vaginal therapeutic delivery device according to another embodiment of the invention.

FIG. 17A is a perspective view of a vaginal therapeutic delivery device according to another embodiment of the invention.

FIG. 17B is a side cross-sectional view of a vaginal therapeutic delivery device according of the embodiment of FIG. 17A.

FIG. 17C is a side cross-sectional disassembled view of a vaginal therapeutic delivery device according of the embodiment of FIG. 17A.

FIG. 18A is a perspective view of a vaginal therapeutic delivery device according to another embodiment of the invention.

FIG. 18B is a side cross-sectional view of a vaginal therapeutic delivery device according of the embodiment of FIG. 18A.

FIG. 19 is a side view, in partial cross-section, of a vaginal therapeutic delivery device according to another embodiment of the invention.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF EMBODIMENTS OF THE INVENTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

U.S. Pat. Nos. 11,083,635 and 11,565,096, as well as pending US Application Publication No. 2020/0323699, are herein incorporated by reference in their entireties.

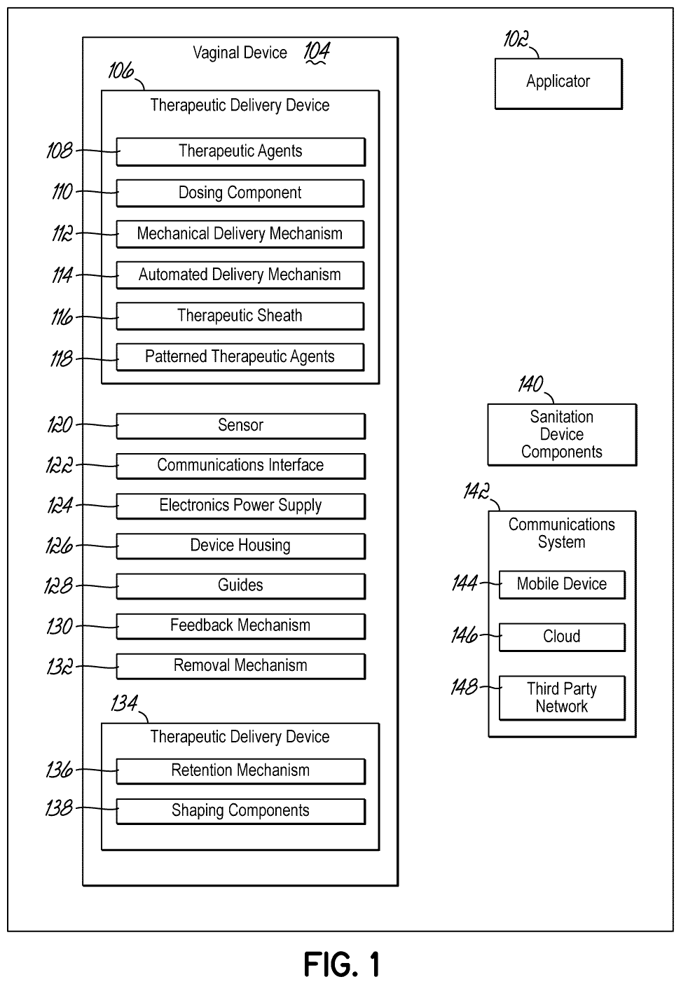

FIG. 1 is a system for a vaginal therapeutic delivery device. This system is comprised of an applicator 102, which is a device for delivering a vaginal device 104 to the treatment site in contact with the walls of the vaginal canal. In an embodiment, the applicator 102 is similar to a tampon applicator. In further embodiments, a traditional tampon applicator can be used to apply a vaginal device 104.

The vaginal device 104 may be placed within the applicator 102 or coupled to the applicator, or may be prepackaged within an applicator 102, and can be inserted into the vaginal opening at which point the applicator 102 may be removed, leaving the vaginal device 104 in the vaginal canal in contact with the mucosa of the vaginal wall. In some embodiments, an applicator 102 may be functional in that is may also deliver therapeutic agents 108. For example, therapeutic agents 108 may comprise single or multiple layers, applied to any of vaginal device 104, applicator 102, or both. For example, an applicator may have one or more therapeutic agent(s) 108, which relieves pain or numbs the tissues which it contacts to provide a pretreatment or treatment when the applicator is inserted into the vaginal canal.

A vaginal device 104 is generally comprised of non-absorbent materials and may have a generally tubular shape, such as a cylindrical shape, a curved and tapered leading end, and a cylindrical outer surface. The vaginal device 104 may additionally comprise a therapeutic delivery device 106 for administering at least one therapeutic agent 108 via the mucosa of the vaginal canal and may additionally comprise one or more of a device housing 120, a therapeutic sheath 116, sensors 120, communications interface 122, electronics power supply 124, retention mechanism 136, and a removal mechanism 132. The vaginal implant may fit in a traditional tampon applicator and be inserted into the vaginal canal following a similar procedure to applying a tampon. The vaginal implant may be configured to perform at least one of delivering one or more therapeutic agents 108 or performing a diagnostic function. Delivering a therapeutic agent 108 comprises administering at least one therapeutic agent 108 to the mucosa of the vaginal wall and/or surrounding tissues. Performing a diagnostic function may comprise any of measuring and/or monitoring at least one metric such as temperature, pH, microbiome, etc. via one or more sensors which may additionally or alternatively comprise one or more assays or sampling devices. The vaginal device 104 may additionally perform a function of leakage protection or positional stability, primarily to prevent the discharge of the therapeutic agents 108 during application. In some embodiments, therapeutic agents 108 may be deposited directly on the vaginal device 104. In some embodiments, the vaginal device 104 may be used without the need for an applicator 102.

A therapeutic delivery device 106 is a component of a vaginal device for delivery of a therapeutic agent 108, such as a pharmaceutical, medicinal, or holistic composition. The therapeutic delivery device 106 may comprise a therapeutic agent 108, dosing component 110, mechanical delivery mechanism 112, automated delivery mechanism 114, and a therapeutic sheath 116, and patterned therapeutic agents 118. Each component of a therapeutic delivery device 106 may be optional depending on the configuration of the vaginal device 104 such as whether the device is being used to perform a diagnostic function or the type of therapeutic agent 108 being delivered.

A therapeutic agent 108 is any of a pharmaceutical, medicinal, or holistic composition which may be comprised of one or more active ingredients including pharmaceutical, medicinal, or holistic components. A therapeutic agent 108 may be formulated to achieve a desired result, such as to treat menstrual pain, treat an infection, etc. The therapeutic agent 108 may take any form as indicated by the most efficient or effective delivery method such as a solid suppository, a fluid, a foam, a cream, a vapor, gas, a gel, mixtures or combinations of states of matter, etc. The therapeutic agent 108 may comprise pharmaceutical, medicinal, or holistic compositions available without a prescription from a doctor such as over the counter or may require a doctor's prescription. Similarly, the therapeutic agent 108 may be intended to be used under the guidance of a medical professional or may otherwise be available for users to use at their own discretion. The therapeutic agent 108 may additionally comprise a lifestyle composition such as a deodorizer, moisturizer, lubricant, etc. to perform functions not necessarily related to the user's health, but instead may improve the user's quality of life or experience.

Pharmaceutical therapeutic agents 108 may comprise any one or more of Antipyretics, Analgesics, Antimalarials, Antibiotics, Mood Stabilizers, Hormone Replacements, Stimulants, Tranquilizers, Statins, Antacids, Reflux Suppressants, Antiflatulents, Antidopaminergics, Proton Pump Inhibitors (PPIS), H2-Receptor Antagonists, Cytoprotectants, Prostaglandin Analogues, Laxatives, Antispasmodics, Antidiarrhoeals, Bile Acid Sequestrants, Opioids, B-Receptor Blockers (“Beta Blockers”), Calcium Channel Blockers, Diuretics, Cardiac Glycosides, Antiarrhythmics, Nitrates, Antianginals, Vasoconstrictors, Vasodilators, ACE Inhibitors, Angiotensin Receptor Blockers, Beta-Blockers, A Blockers, Thiazide Diuretics, Loop Diuretics, Aldosterone Inhibitors, Coagulation, Anticoagulants, Heparin, Antiplatelet Drugs, Fibrinolytics, Anti-Hemophilic Factors, Haemostatic Drugs, HMG-Coa Reductase Inhibitors, Hypolipidaemic Agents, Anaesthetics, Antipsychotics, Antidepressants (Including Tricyclic Antidepressants, Monoamine Oxidase Inhibitors, Lithium Salts, And Selective Serotonin Reuptake Inhibitors (SSRIS)), Antiemetics, Anticonvulsants/Antiepileptics, Anxiolytics, Barbiturates, Movement Disorder (E. G., Parkinson's Disease) Drugs, Stimulants (Including Amphetamines), Benzodiazepines, Cyclopyrrolones, Dopamine Antagonists, Antihistamines, Cannabinoids, 5-HT (Serotonin) Antagonists, Analgesic Drugs, Nsaids (Including COX-2 Selective Inhibitors), Muscle Relaxants, Neuromuscular Drugs, Anticholinesterases, Bronchodilators, Antitussives, Mucolytics, Decongestants, Corticosteroids, Beta2-Adrenergic Agonists, Anticholinergics, Mast Cell Stabilizers, Leukotriene Antagonists, Androgens, Antiandrogens, Estrogens, Gonadotropin, Corticosteroids, Human Growth Hormone, Insulin, Antidiabetics (Sulfonylureas, Biguanides/Metformin, Thiazolidinediones, Insulin), Thyroid Hormones, A, Ntithyroid Drugs, Calcitonin, Diphosponate, Vasopressin Analogues, Alkalinizing Agents, Quinolones, Cholinergics, Antispasmodics, 5-Alpha Reductase Inhibitor, Selective Alpha-1 Blockers, Sildenafils, Fertility Medications, Hormonal Contraception, Ormeloxifene, Haemostatic Drugs, Antifibrinolytics, Hormone Replacement Therapy (HRT), Bone Regulators, Beta-Receptor Agonists, Follicle Stimulating Hormone, Luteinising Hormone, LHRH, Gamolenic Acid, Gonadotropin Release Inhibitor, Progestogen, Dopamine Agonists, Oestrogen, Prostaglandins, Gonadorelin, Clomiphene, Tamoxifen, Diethylstilbestrol, Antifungals, Antileprotics, Antituberculous Drugs, Antimalarials, Anthelmintics, Amoebicides, Antivirals, Antiprotozoals, Probiotics, Prebiotics, Vaccines, Immunoglobulins, Immunosuppressants, Interferons, Monoclonal Antibodies, Anti-Allergics, Antihistamines, Corticosteroids, Electrolytes, Mineral Preparations (Including Iron Preparations And Magnesium Preparations), Parenteral Nutritions, Vitamins, Anti-Obesity Drugs, Anabolic Drugs, Haematopoietic Drugs, Food Product Drugs, Cytotoxic Drugs, Therapeutic Antibodies, Sex Hormones, Aromatase Inhibitors, Somatostatin Inhibitors, Recombinant Interleukins, G-CSF, Erythropoietin.

Medicinal therapeutic agents 108 may comprise any one or more of Antihistamine Drugs (including the prescription drugs Clarinex and Xyzal and OTC medicines Allegra, Benadryl, Claritin, Chlor-Trimeton, Dimetane, Zyrtec and Tavist), Anti-infective Agents (including penicillins and anti-virals), Antineoplastic Agents, Autonomic Drugs, Blood Derivatives, Blood Formation, Coagulation, and Thrombosis Agents, Cardiovascular Drugs (including digoxin, acebutolol, propranolol and lisinopril), Cellular Therapy, Central Nervous System (CNS) Agents (including stimulants and depressants), Contraceptives, Dental Agents, Diagnostic, Electrolytic, Caloric, and Water Balance, Enzymes, Respiratory Tract Agents, Eye, Ear, Nose, and Throat (EENT) Preparations, Gastrointestinal Drugs (including rabeprazole sodium, nitazoxanide, bevacizumab and nizatidine), Gold Compounds, Heavy Metal Antagonists, Hormones and Synthetic Substitutes, Local Anesthetics, Oxytocics, Radioactive Agents, Serums, Toxoids, and Vaccines, Skin and Mucous Membrane Agents, Smooth Muscle Relaxants (including cyclobenzaprine and carisoprodol), Vitamins, Miscellaneous Therapeutic Agents, Pharmaceutical Aids.

Holistic therapeutic agents 108 may comprise any one or more of Ginkgo biloba, Hypericum perforatum, Herbal pollen extract NOS, Senna alexandrina, Herbal extract NOS, Cimicifuga racemose, Echinacea purpurea, Plantago ovata, Serenoa repens, Glycine max, Oenothera biennis, Vitis vinifera, Cannabis sativa, Cannabis indica, Mentha x piperita, Citrus x paradisi, Valeriana officinalis, Silybum marianum, Viscum album, Allium sativum, Vitex agnus-castus, Pelargonium reniforme root, Digitalis purpurea, Ginseng NOS, Humic acid, Vitamin A, B-6, B-12, C, D, E, K, Chromium, Folic acid, Calcium, Iron, Zinc, Plant, Fruit and Nut tinctures, concentrates or extracts.

Other types of Therapeutic agents 108 might include one or more of the following: Dinoprostone, Misoprostol, Boric Acid, Miconazole Nitrate, Polymyyxin B Sulfate, Nystatin, Hyaluronic Acid sodium slat, EDTA, Clotrimazole, Lactobacilli gasser and rhamnosus, Nitric oxide, Dapivirin, Levonorgestrel, Metronidazole, HC4-N, Lactic Acid, Segestrone acetate, Ethinyl estradiol, Estradiol, Etonogestrel, Etongenstrel/Ethinyloestradiol, Sildenafil, THC, Vicodin, Norco, Lortab, Percocet, Oxycodone, Hydrocodone, Codine, Dilaudid, Opana, Fentanyl, Lidocaine, Tramadol, Pregabalin, Duloxetine, Gabapentine, Tramadol, Topramate, Nortriptyline, Aspirin, Butrans, Naxopren, Povidone Iodine, Natamycin-gamma Cyclondextrin, ketoconazole, Ketomine, Intraconazole, Hexyl-aminolevulinate, Oxbutynin, Curcumin, Clindamycin hydrochloride, Nystatin, Nonoxynol-9, Anti-sperm antibodies (ASA) on Film polyvinyl alcohol-based water, soluble contraceptive film, Anti-HIV (VRC01-N), Anti-Film Herpes Simplex Virus (HSV8-N) mAb, metronidazole, nitric oxide, Levonorgestrel, Nonoxynol-9 Benzalkonium chloride, Anti-HIV microbicides, PHI-113, PHI-346, and PHI-443.

A dosing component 110 is any component which functions to dose the amount of therapeutic agent 108 to be administered to a user. A dosing component 110 may comprise a reservoir for storing a therapeutic agent 108. A dosing component 110 may additionally comprise a dosing pump. A dosing component 110 may alternatively refer to a therapeutic sheath 116 which can be exposed (fully or partially) or torn to reduce the dose delivered to less than the maximum dose. Likewise, a dosing component 110 may refer to the results of any equipment used to deposit a therapeutic agent 108 on a therapeutic sheath 116, vaginal device 104, applicator 102, etc. such as a printing device which may include a 3D printer. The dosing component 110 may additionally refer to an activation device 122, mechanical delivery mechanism 112 or automated delivery mechanism 114 where a pre-determined or selected dosage of one or more therapeutic agent(s) 108 is dispensed, ejected, or otherwise delivered into the vaginal canal of the user.

A mechanical delivery mechanism 112 is a delivery mechanism which carries and administers at least one therapeutic agent 108 to the mucosa of the vaginal canal or surrounding tissues. Mechanical delivery system 112 may also include therapeutic agents 108 to be contained in a rupturable container, which when deployed disrupts the container to dispense therapeutic agents 108. Mechanical delivery system 112 may further include a combination of the therapeutic agent 108 and a reacting agent (which may be another therapeutic agent 108) that allows for improved efficacy of the system. In its simplest form, the mechanical delivery mechanism 112 may hold a suppository to facilitate its insertion into the vaginal canal which is released upon removal of the applicator 102. In other embodiments, a mechanical delivery mechanism 112 may comprise a delivery mechanism for actively dispensing a therapeutic agent 108 from a reservoir within the therapeutic delivery device 106. In some embodiments, the mechanical delivery mechanism 112 may be activated by the removal of the applicator 102 after insertion of the vaginal device 104 into the vaginal canal. In other embodiments, the mechanical delivery mechanism 112 may be activated by another chemical or kinetic activation source. For example, mechanized mechanical delivery systems might include a pipette, a pump, a syringe, or may take the form of a flexible package to be squeezed.

An automated delivery mechanism 114 is a delivery mechanism which carries and administers at least one therapeutic agent 108 to the mucosa of the vaginal canal or surrounding tissues via an at least partially automated means. For example, the automated delivery mechanism 114 may be activated by a sensor 120 or command from a mobile device 144. In some embodiments, an automated delivery mechanism 114 may integrated with and/or be a mechanical delivery mechanism 114 which is not manually activated. For example, a mechanical delivery mechanism 112 which activates upon removal of an applicator 102 may be considered an automated delivery mechanism 114. Alternatively, a mechanical delivery mechanism 112 which requires a manual user operation, such as twisting or pulling on a part of the applicator 102, vaginal device 104, etc. would be considered a mechanical delivery mechanism 114 but not an automated delivery mechanism 114. In some embodiments, an automated delivery mechanism 114 may respond to an observed condition, such as a low humidity measurement from a sensor 120, or an increased temperature, or electromyography (EMG) activity indicative of pain which may automatically release an analgesic.

A therapeutic sheath 116 may be applied to at least a portion of the cylindrical outer surface of the vaginal device. The therapeutic sheath 116 may comprise a sheath of impermeable film material which completely envelops the vaginal device 104 or may instead only partially cover the vaginal device 104, such as in a delivery area or section. In some embodiments, the therapeutic sheath 116 may be optionally used and the insertable vaginal device may just be a physical barrier after a dosage of a therapeutic agent is delivered to the vaginal cavity. The therapeutic sheath 116 may be at least partially coated in a therapeutic agent 108 and may additionally be enclosed within a water-soluble film to prevent the therapeutic agent 108 from being displaced during application of the vaginal device 104 or to delay the release of the therapeutic agent 108. In some embodiments, the therapeutic agent 108 may be combined with the water-soluble film or a similar composition to facilitate an extended release of the therapeutic agent 108. In some embodiments, a therapeutic sheath 116 may be used with and/or deployed by an applicator 102.

A patterned therapeutic agent 118 is a deposition or extrusion of one or more therapeutic agents 108 in a specific pattern. The pattern may be regular or repeating, or it may appear to be random, being based instead on an algorithm to deliver a specific amount of one or more therapeutic agents 108 to the vaginal canal at a specific rate. The rate and quantity of therapeutic agent 108 released is a function of the rate of dissolution of the materials used, including the therapeutic agent 108 and any binders which may be used, and the surface area of the therapeutic agent 108 as the therapeutic agent 108 comprising the patterned therapeutic agent 118 dissolves. In an embodiment, the patterned therapeutic agent 118 may be a series of shapes, such as dots or circles, which repeat in a range of sizes, frequencies, height, etc. In some embodiments, the patterned therapeutic agent 118 may be a gradient. The patterned therapeutic agent 118 may further comprise any shape including polygons, lines, etc. in any arrangement or orientation. In still further patterns, one agent might be positioned at one position along the length of the vaginal device and one or more different agents might be positioned at other positions along the length of the vaginal device. Alternatively, the quantity of therapeutic agent 108 might be delivered in stacked layers on a device, that dissolve or are exposed in sequence so that the agents are released in a sequential fashion.

A sensor 120 is a device for measuring one or more metrics or characteristics of a target subject. One or more sensors 120 may be integrated into the vaginal device 104 to measure or monitor one or more metrics such as temperature, pH, or the presence of a pathogen. In some embodiments, a sensor 120 may be a chemical indicator such as a pH strip which changes color when in the presence of fluids. In other embodiments, a sensor 120 may be a digital device which may transmit data to a connected mobile device 144 via a communications interface 122. In further embodiments, a sensor 120 may be an assay. In some embodiments, sensors 120 may be modular, such as being changeable similar to a replaceable therapeutic delivery device 106. In some embodiments, a sensor 120 may detect when a diagnostic function has been completed or a therapeutic agent 108 has been fully administered and/or absorbed which may be communicated to a mobile device 144 or may alternatively provide an indication via another means including via a feedback mechanism 130 such as a vibration, electrical stimulation, etc.

A communications interface 122 enables communication with one or more mobile devices 144, networks or a cloud 146. A communications interface 122 may use one or more wireless protocols such as Wi-Fi, Bluetooth, LTE, 4G, 5G, near field communication, etc. to communicate with a mobile device 144 or cloud 146. In some embodiments, a communications interface 122 may be a physical interface such as an ethernet or USB port. An electronics power supply 124 is a device for supplying electricity to a vaginal device 104. An electronics power supply 124 may be a single use or rechargeable battery. Alternatively, an electronics power supply 124 may receive power wirelessly such as via wireless power transfer. An example of wireless power transfer utilizes inductance such as the Qi charging standard. In further embodiments, the electronics power supply 124 may be a power cord connected to the vaginal device 104.

A device housing 126 provides the structure and protection for the vaginal device 104 and its components. The device housing 126 comprises a bio-compatible material such as silicone, nitrile, etc. and fully encloses the components within the vaginal device 104 and/or therapeutic delivery device 106. The device housing 126 may additionally provide the structure of the vaginal device 104 and/or therapeutic delivery device 106. In some embodiments, the device housing 126 may be comprised of an inner part which does not need to be constructed of bio-compatible materials and an outer portion designed to contact the body.

Guides 128 may refer to physical features on an applicator 102 or vaginal device 104 to aid in the proper method and placement of the vaginal device 104 into the vaginal canal. Guides 128 may include grips, markings, bumps, ridges, etc. intended to help the user grip, move, and position the applicator 102 and/or vaginal device 104 to the correct location to ensure the proper delivery of at least one therapeutic agent 108, or alternatively to accurately perform a diagnostic function via one or more sensors 120. In some embodiments, guides 128 may refer to documentation and/or methods of making documentation available to a user such as via a mobile device 144. In some embodiments, a QR code, barcode, or link may be present on the applicator 102, vaginal device 104, or packaging for the applicator 102 and/or vaginal device 104, which links to documentation including procedures, videos, etc. to instruct and aid the user in the correct use and placement of the vaginal device 104. In some embodiments, guides 128 may refer to dosage or treatment guides 128 to aid the user in selecting the correct therapeutic agents 108 and the correct dosage for the therapeutic agents 108. In some embodiments, the guides 128 may refer the user to consult a medical professional.

A feedback mechanism 130 is a component which provides feedback to the user. In some embodiments, a feedback mechanism 130 may include bumps, ridges, and other physical features of the applicator 102, vaginal device 104, device housing 126, etc. for aiding in the correct placement of the vaginal device 104. In other embodiments, the feedback mechanism 130 may be an active feature such as a haptic device, providing vibration, or alternatively an electrical device providing electrical stimulation, to provide an indication or notification to the user. For example, the feedback mechanism 130 may vibrate to indicate that the therapeutic agent 108 has been delivered and the device can be removed. In an alternate embodiment, the feedback mechanism 130 may provide an electrical stimulation to the user indicating that the device should be removed immediately due to a misplacement of the vaginal device 104, or some other error condition. In other embodiments, a feedback mechanism 130 may be a sound generating device. The feedback mechanism 130 may instead work via communication with a mobile device 144 via a communications interface 122.

A removal mechanism 132 is an element or feature allowing the vaginal device 104 to be easily removed from the vaginal canal. In some embodiments, removal mechanism 132 may deliver therapeutic agent 108 via tubing, which may be comprised of silicone or other polymeric materials. In a more specific embodiment, removal mechanism 132 comprises tubing with microchannels to promote delivery of therapeutic agent 108. In the simplest embodiments, the removal mechanism 132 may be a string secured to the vaginal device 104. In other embodiments, the removal mechanism 132 may additionally perform the function of deactivating a retention mechanism 136 or may actuate a therapeutic delivery device 106 such that a therapeutic agent 108 is released. The removal mechanism 132 may additionally comprise an indicator to indicate to the user that the vaginal device 104 has completed its intended function and can be removed. Personalization device components 134 facilitate customization of a vaginal device 104 to the user or a specific use.

Personalization may include adjusting the shape, size, therapeutic agents 108, sensors 120, etc. Personalization device components 134 may include a retention mechanism, which may expand, contract, or otherwise conform to the vaginal canal of a user to improve fit of a vaginal device 104 to improve comfort and prevent the vaginal device 104 from moving during use. Personalization device components 134 may refer to shaping components 138, such as adjustable, articulating, inflating, molding, etc. components which can be altered to conform to the vaginal canal of a user. In some embodiments, personalization device components 134 may be adjusted outside the body prior to inserting into the vaginal canal. In other embodiments, personalization device components 134 may be adjusted during or after insertion into the vaginal canal. In some embodiments, personalization device components 134 may comprise the use of a perfume, deodorizer, etc. chosen by the user.

A retention mechanism 136 is a mechanism or structure design of a vaginal device 104 which may either hold the vaginal device 104 in a desired location within the vaginal canal or prevent the discharge of fluids, primarily to prevent therapeutic agents 108 from escaping the vaginal canal. In some embodiments, the retention mechanism 136 is static, which may be comprised of a flare at the base of the vaginal device 104, or may alternatively be a deployable mechanism, such as a balloon which may be inflated upon insertion to provide a seal at the entrance of the vaginal canal. Shaping components 138 are any components which allow for the shape of the vaginal device 104, or specifically the device housing 126 of the vaginal device 104, to be adjusted, molded, shaped, etc. to improve the comfort, fit, function, etc. of the vaginal device 104. Shaping components 138 may be adjusted before, during, or after insertion of the vaginal device 104 into the vaginal canal. In some embodiments, the shaping components may aid in evenly distributing the pressure of a vaginal device 104 against the wall of the vaginal canal aiding in even contact and delivery of a therapeutic agent 108 via a therapeutic sheath 116. In some embodiments, the therapeutic sheath 116 may stretch or otherwise expand to conform to the new shape of a vaginal device 104 which has been altered via one or more shaping components 138. In some alternate embodiments, shaping components 138 may refer to one or more accessory devices which may be used to manipulate the shape of a vaginal device 104 which is not itself a component of the vaginal device 104, such as a mandrel or form to help bend or mold the vaginal device 104.

Sanitation device components 140 refer to any device, component, article, etc. which may be used to help sanitize, or aid sanitization or sanitary use of an applicator 104, vaginal device 104, therapeutic delivery device 106, etc. Sanitation device components 140 may refer to disposable articles such as a therapeutic sheath 116, gloves, bags, adapters, applicators 102, etc. In some embodiments, sanitation device components 140 may refer to reusable components, such as a sanitation device for cleaning reusable components such as a vaginal device 104. In some embodiments, sanitation device components 140 may refer to commonly used cleaning materials and articles such as soap, water, a sponge, cloth, paper towel, etc. for washing articles. Sanitation device components 140 may refer to sources of ultraviolet light, an autoclave or other source of heat sufficient to disinfect materials, applicator of antibacterial soaps or substances, etc. Sanitation device components 140 may additionally refer to materials with naturally antibiotic properties, such as a material impregnated with silver. In such embodiments, the sanitation device components 140 may be integrated into the vaginal device 104 or its components. A communication system 142 is a network of connected devices.

A communications system 142 comprises at least one communication protocol which may include any of Wi-Fi, Bluetooth, NFC, 4G, 5G, LTE, etc. and may additionally utilize wired connections such as ethernet, USB, or commercial telecommunications networks to include both wired interchanges and cellular and satellite communications networks. A communications system 142 may be comprised of at least two devices or resources comprising any of mobile devices 144, clouds 146, 3rd party networks 148, and vaginal devices 104 and other connected devices which include a communications interface 122 to facilitate communication within the communications system 142.

A mobile device 144 is a computing device which may be used by the user of a vaginal device 104 or alternatively by a medical professional to receive data from a vaginal device 104 via a communications interface 122. The mobile device 144 may be any of a cell phone, tablet, computer, or other proprietary device. In some embodiments, the mobile device 144 may receive data by recording audio from the user or sounds emitted by the vaginal device 104. In other embodiments, the mobile device 144 may receive data via a camera, such as taking a picture or video of a pH strip. An app on the mobile device 144 may be used to interpret the image, such as to determine the pH measured by the vaginal device 104. In other embodiments, the image taken may be of an assay or the therapeutic delivery device 106 to verify that the therapeutic agent 108 was properly applied. The mobile device 144 may receive a notification, alert, or instruction from the vaginal device 104 when it has completed its intended function and is ready to be removed. In some embodiments, the mobile device 144 may function as a timer, such that the user indicates when the vaginal device 104 was inserted and the mobile device 144 provides a notification when a suitable amount of time has elapsed and the vaginal device 104 should be removed.

A cloud 146 is a distributed network of computational or data storage resources. A cloud 146 may be a private cloud, where access is restricted via encryption or is physically separate from other networks such as the internet. In other embodiments the cloud 146 may be a public cloud accessible via the internet. In some embodiments, a public cloud may additionally be secured via encryption. Typically, a public cloud utilizes resources from third party providers.

A 3rd party network 148 is a proprietary network comprised of private resources which may be made available based upon registration and/or agreement to the private party's terms of service. In an embodiment, a 3rd party network 148 may comprise one or more servers, databases, application, etc. operated by a 3rd party to provide additional functionality to a vaginal device 104, such as tracking data measured by one or more sensors 120, tracking activity such as how often a therapeutic agent 108 was used, the dosage, and the reason for treatment. In some embodiments, a 3rd party network 148 may be a cloud 146 resource provider such as Microsoft Azure, Amazon Web Services, etc. used to host one or more applications. A 3rd party network 148 may include algorithms to calculate and notify or execute future methods for analyzing data from the vaginal device. The algorithms may include (1) safety algorithms, (2) timing algorithms, (3) dosing algorithms, (4) feedback algorithms, etc.

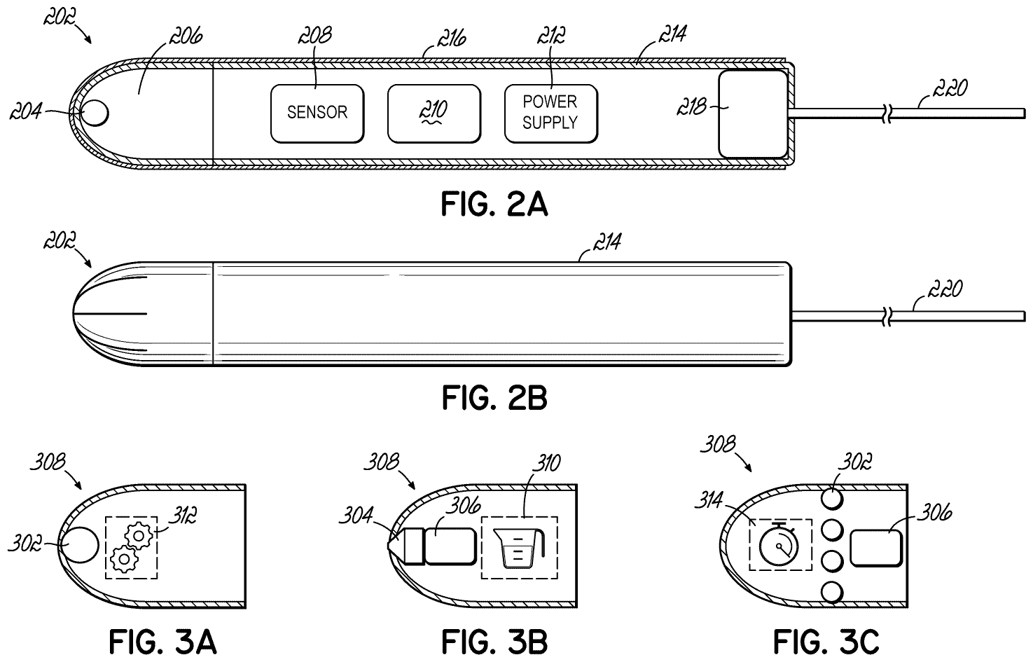

The functioning of the “vaginal device” will now be explained with reference to FIG. 2. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

This figure displays the vaginal device 104. The vaginal device 104 comprises a vaginal device 202 is an article suitable for insertion into the vaginal canal for delivery of a therapeutic agent 204, or to position one or more sensors 208 so as to measure or monitor one or more metrics related to the health of the user. Vaginal device 202 may contain any of the components or combination of components in this invention. The vaginal device 202, when properly inserted, contacting the mucosal membrane of the vaginal canal. The vaginal device 202 may function as a diagnostic device using digital sensors connected to a mobile device 144 via a communications interface 210 or may alternatively comprise one or more physical testing devices such as pH indicators, assays, or sampling devices for further testing and analysis outside the body.

An app running on a mobile device 144 may be used to further analyze such physical testing devices to interpret or confirm a result. The vaginal device 202 may alternatively or additionally administer one or more therapeutic agents 204 in one or more formats such as a solid suppository, fluid, cream, gel, etc. The therapeutic agent 204 may simply be inserted for immediate application or may be designed for delayed or extended release or some combination thereof. The vaginal device 202 may additionally perform the function of leakage protection or positional stability via a retention mechanism 218, primarily to facilitate the effective application of one or more therapeutic agents 204. The vaginal device 202 is largely comprised of bio-safe materials such as silicone or nitrile, however the interior materials not in contact with the body or bodily fluids may be made of other non-toxic materials including metal, plastic, semiconductors, etc.

A therapeutic agent 204 comprises one or more pharmaceutical, medicinal, or holistic components or compositions which may be administered via contact with the mucosa of the vaginal canal or the surrounding tissues. The therapeutic agent 204 may take any appropriate form to facilitate effective treatment of one or more conditions including menstrual pain, endometriosis, dysmenorrhea, etc. The therapeutic agent 204 may additionally treat localized symptoms such as balancing pH, reducing temperature or inflammation, etc. Exemplary therapeutic agents 204 may include an analgesic such as ibuprofen, a medicinal such as CBD or CBDA, or probiotics such as those of the genus lactobacillus. Exemplary forms of a therapeutic agent 204 may include solid suppositories, liquids or foams which may be dispensed via a nozzle or ejection orifice, or a cream or gel which may be disposed on a therapeutic sheath 216 which may be coated with a water-soluble film to ensure proper dosing and positioning of the therapeutic agent 204. In some embodiments, the therapeutic agent 204 may comprise an antibiotic to treat ailments such as urinary tract or yeast infections. In other embodiments, the therapeutic agent 204 may comprise a moisturizer or medicant to improve vaginal dryness.

In other embodiments, the therapeutic agent 204 may comprise a deodorant or perfume formulated for application to the tissues of the vaginal canal, or alternatively to be released via an inserted vaginal device 202. In such embodiments, the effect may be performed via a therapeutic delivery device 206 while inserted in the vaginal canal, while generally avoiding direct contact with the mucosa and surrounding tissues of the vaginal canal. In some embodiments, the therapeutic agent 204 may perform actions such as numbing the walls of the vaginal canal and surrounding tissues, or alternatively increasing the sensitivity of the vaginal canal and surrounding tissues, particularly in anticipation of intimacy, or alternatively a medical procedure. In some embodiments, the therapeutic agent 204 may comprise a lubricant or moisturizer. In some embodiments, different therapeutic agents 204 may be combined in the same treatment within therapeutic delivery device 206. The different therapeutic agents 204 may be used in combination with each other simultaneously or may be used to treat in succession for a desired effect. For example, a numbing agent may be immediately delivered, whereas a form of antibiotic may be time-released through a slower to dissolve film. Both therapeutic agents 204 may be disposed within therapeutic delivery device 206 to allow for one step application and use.

A therapeutic delivery device 206 comprises any mechanism, feature, or component of a vaginal device 202 which contains and/or dispenses one or more therapeutic agents 204. For example, a therapeutic delivery device 206 may comprise a tablet holder for securing a suppository for insertion into the vaginal canal. In some embodiments, the suppository may be held in place by an applicator 222 and is otherwise released when the applicator 222 is removed following insertion of the vaginal device 202 into the vaginal canal. The therapeutic device 206 may alternatively comprise a therapeutic reservoir for containing a fluid which may be dispensed upon removal of the applicator 222 following insertion of the vaginal device 202 into the vaginal canal, or which may be administered via other means, such as manual actuation via external means, automated delivery which may be controlled from a mobile device 144 via a communications interface 210, or by the dissolution of a water-soluble material or plug which may release the contents of a therapeutic reservoir under a small amount of pressure, likely via a mechanical spring affixed to a syringe type plunger. In further embodiments, the therapeutic delivery device 206 may comprise a therapeutic sheath 216, comprising a removable, non-porous film material which may be partially or wholly coated with one or more therapeutic agents 206.

Therapeutic agents 204 disposed upon a therapeutic sheath 216 may further be mixed into or encapsulated by a water-soluble material to delay administration of the therapeutic agent 204 at least until the vaginal device 202 is properly positioned within the vaginal canal and in some embodiments, designed for more precise time-release of therapeutic agent 204. In some embodiments, the therapeutic delivery device 206 may be controlled or authorized to release a therapeutic agent 204 by a medical professional either local to or remote from the user. In other embodiments, the medical professional may provide guidance to the user who may adjust the configuration of the vaginal device 202 or therapeutic delivery device 206 to facilitate the administration of a treatment such as applying a therapeutic sheath 216 prepared with a specific therapeutic agent 204 and likewise a specific dosage of the therapeutic agent 204.

A sensor 208 may comprise a digital device or a physical test for measuring or monitoring at least one metric related to the health of the user. In an exemplary embodiment, the sensor 208 may be a digital temperature sensor for measuring the temperature of the vaginal canal and surrounding tissues. In additional embodiments, the sensor 208 may comprise a humidity or pH sensor. In some embodiments, the sensor 208 may be a physical device such as a pH indicator, assay, or sampling device for external interpretation and/or analysis which may be aided by a mobile device 144. Sensor 208 may also detect near-infrared light, and undetectable visible light, based on a photodiode and holograms and may sense many changes in biotechnology. The sensor 208 may permanently installed within or upon the vaginal device 202 or may be modular, capable of being swapped with one or more sensors or therapeutic delivery devices 206. In an embodiment, a sensor 208 may be a pH indicator integrated into or attached to a therapeutic sheath 216. In such embodiments, the therapeutic sheath may not include a therapeutic agent 204 or may be partially coated with a therapeutic agent not including the surface area occupied by the pH indicator. In further embodiments, a sensor 208 may sample fluids or tissue from the vaginal canal which may be tested externally via a secondary device which may additionally utilize a mobile device 144 to interpret and communicate the results of the test.

In some embodiments, a sensor 208 may communicate directly with a mobile device 144 in possession of a medical professional via a communications interface 210 and the cloud 146, or indirectly via communication of the vaginal device 202 with a user's mobile device 144 which may then relay data from the one or more sensors 208 to a mobile device 144 in possession of a medial professional.

A communications interface 210 may comprise any means facilitating the communication of a vaginal device 202 with the cloud 146 or one or more authorized mobile devices 144. The communications interface 210 may comprise a wireless interface comprising at least an antenna and transmitter, or a physical connection, such as a wire which may interface via a port such as an ethernet or USB port or be permanently attached. In further embodiments, the communications interface 210 may facilitate bidirectional communications allowing for sensor 208 data to be transmitted to a cloud 146 or mobile device 144 or may alternatively receive instructions from a cloud 146 or mobile device 144 such as a command to release a therapeutic agent 204, particularly at an indicated rate. Likewise, a communications interface 210 may be used to provide an indication to a mobile device 144 that administration of the therapeutic agent 204 or the diagnostic function of the vaginal device 202 is complete and that the vaginal device 202 can be removed. Such features may help to prevent devices from being inserted for too long which may have adverse effects potentially culminating in toxic shock syndrome. This is further avoided by constructing the vaginal device 202 with nonabsorbent, biosafe materials.

An electronics power supply 212 may comprise any means facilitating the delivery of power to one or more components within a vaginal device 202. In an embodiment, an electronics power supply 212 may comprise a rechargeable or non-rechargeable battery. An example of a rechargeable battery is a lithium ion or nickel metal hydride battery. An example of a non-rechargeable battery is an alkali or lead acid battery. While it is preferred that the electronics power supply 212 be constructed of biosafe materials, it may not be required as the electronics power supply 212 should be isolated from contact with the user via a sealable device housing 214. In some embodiments, a rechargeable battery may be recharged via a physical port comprised of a barrel jack or USB port or may preferably charge via a wireless charging method such as using inductance via the Qi charging standard. In some embodiments, the electronics power supply 212 may comprise an internal and external component to facilitate wireless power transfer such as via inductance wherein a first coil is placed within the vaginal device 202 and a second coil is positioned outside the user's body in proximity to the vaginal device 202 such that a current applied to the second coil may generate a magnetic field which then induces a current in the first coil which can be used to power the vaginal device 202. In some embodiments, the electronics power supply 212 may be located externally to the user such as a battery or external power interface connected to the vaginal device 202 via a power cable. In ideal embodiments, the electronics power supply 212 provides a voltage to the vaginal device 202 of between 1.5 to 12 volts direct current.

A device housing 214 may be at least partially comprised of biocompatible materials. In an ideal embodiment, the surfaces of the device housing 214 in contact with the mucosa of the vaginal canal of the user are comprised of biocompatible materials such as silicone. It is preferable that these materials are nonpermeable and easy to clean if the vaginal device 202 is intended to be reused. In some embodiments, the device housing 214 may be a completely sealed device to prevent intrusion of bodily fluids into the vaginal device 202. In other embodiments, the device housing 214 may be partially sealed such that some components, such as a sensor 208 or therapeutic delivery device 206 may be constantly or selectively in contact with the mucosa of the vaginal canal and/or surrounding tissues to facilitate measurement and/or monitoring of one or more metrics related to the user's health and/or administration of one or more therapeutic agents 204. In some embodiments the therapeutic agent 204 may be applied directly to the device housing 214.

A therapeutic sheath 216 is a thin material such as a film which may partially or completely cover a vaginal device 202. In an embodiment, the therapeutic sheath 216 may be used to physically isolate the vaginal device 202. In some embodiments, the therapeutic sheath 216 may be partially or completely coated in a therapeutic agent 204. The therapeutic agent 204 may further be encapsulated in a water-soluble film for temporarily isolating the therapeutic agent 204 at least until the vaginal device 202 is properly positioned within the vaginal canal, the applicator 222 removed, and the therapeutic sheath 216 is placed in contact with the mucosa of the vaginal canal and surrounding tissues. The therapeutic sheath 216 and the distribution of therapeutic agent 204 across its surface may facilitate dosing amounts and rate of release. In some embodiments the therapeutic sheath 216 may be physically altered, such as by tearing a portion of the therapeutic sheath 216, to customize the dosage of therapeutic agent 204 being administered.

A retention mechanism 218 is a feature of a vaginal device 202 which may perform any function related to maintaining the proper position of the vaginal device 202 in the vaginal canal of the user during use, or which may aid retention of therapeutic agent 204 during use of the vaginal device 202. In ideal environments, the fluid retention function should be temporary, for short term use, primarily related to preventing therapeutic agents 204 from being discharged from the vaginal canal prior to completion of a treatment. In some embodiments, the retention mechanism 218 may be a physical feature, such as the flaring out of the base of the vaginal device 202 creating a graduated, somewhat conical, shape. In other embodiments, the retention mechanism 218 is deployable, such as by inflating a pneumatic balloon, or otherwise actuating a mechanism which may be spring loaded or ratcheting. In ideal embodiments, such a mechanism would exert light pressure on the vaginal canal to provide a secure fit, preventing the vaginal device 202 from inadvertently being removed from the vaginal canal. In some embodiments, the retention mechanism 218 may comprise a texture or shape to conform to the walls of the vaginal canal. In some embodiments, the retention mechanism 218 may provide the ability to conform to the vaginal canal of the user, such as by inflating a pneumatic balloon, allowing the shape of the vaginal device 202 to be increased and molded to better fit the vaginal canal of the user. In such embodiments, the retention mechanism 218 may extend along part, most, or all the length of the vaginal device 104 to ensure a proper fit. Such expansion may allow for firm contact between a therapeutic sheath 216 and the mucosa of the vaginal canal.

A removal mechanism 220 is any device, component, or mechanism intended to be used to remove a vaginal device 202 from the vaginal canal of a user. In its simplest form, a removal mechanism 220 may be string securely attached to the vaginal device 202. In some embodiments, such as string may be comprised of nonabsorbent materials which may further be sanitized to facilitate reuse of the vaginal device 202. In some embodiments, the removal mechanism 220 or string may be capable of being replaced between uses of the vaginal device 202. In further embodiments, where a wired communication interface 210 and/or electronics power supply 212 is utilized, the wire facilitating communication and/or power transfer may be used in place of a string as a removal mechanism 220. In some embodiments, removal mechanism 220 is designed to deliver therapeutics agents 204 via a conduit such as microchannels in polymeric tubing. In some embodiments, the removal mechanism 220 may perform the function of releasing a deployed retention mechanism 218. For example, a removal mechanism 220 may allow air to escape from a pneumatic balloon retention mechanism 218, allowing for removal of the vaginal device 202. In alternate embodiments, the removal mechanism 220 may release the clutch of a ratcheting retention mechanism 218 or may compress one or more springs to reduce pressure from the retention mechanism 218 from the walls of the vaginal canal facilitating removal of the vaginal device 202. An applicator 222 is a device for use in inserting a vaginal device 202 into the vaginal canal of the user. In some embodiments, the applicator 222 may be a tampon applicator or may be similar to existing tampon applicators.

An applicator 222 may temporarily house a vaginal device 202. In some embodiments, the vaginal device 202 may be preinstalled in an applicator 222. In such embodiments, the assembly of the vaginal device 202 preinstalled in an applicator 222 may be sealed within a packaging. In other embodiments, the vaginal device 202 may be manually installed into the applicator 222 by the user. The applicator 222 aids in ease of use and proper placement of the vaginal device 202 in the vaginal canal. In some embodiments, the applicator may comprise features which may activate the vaginal device 202 when inserted into the vaginal canal and the applicator 222 is removed. For example, the applicator may peel away or otherwise remove a film covering a therapeutic on a therapeutic sheath 216 or therapeutic delivery device 206. In another embodiment, an applicator 222 may deliver a separate therapeutic agent 204, such as a pre-treatment prior to any therapeutic agent 204 contained in vaginal device 202. In alternate embodiments, the applicator 222 may communicate with a switch on the vaginal device, such as a physical or magnetic switch such that when the applicator is removed, the vaginal device 202 is activated which may include powering on or activation of one or more sensors 208. The applicator 222 may be disposable, ideally comprised of biodegradable or recyclable materials, or may be reusable.

Functioning of the “therapeutic delivery device” will now be explained with reference to FIG. 3. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

This figure displays the therapeutic delivery device 106. A therapeutic delivery device 106 comprises a therapeutic agent 302 is one of any composition with active ingredients belonging to the categories of a pharmaceutical, medicinal, or holistic which are suitable for administration to the mucosa of the vaginal canal or surrounding tissues. A therapeutic agent 302 may comprise one or more active ingredients and may take any appropriate form, such as a solid, as in the case of a suppository, a liquid, such as may be administered as a spray, foam, or secretion via a nozzle 304, or a cream or gel which may be administered via a therapeutic sheath 116. It should be noted that the examples provided herein are representative but not exhaustive of therapeutic compositions and delivery methods and should not be interpreted as limiting.

A nozzle 304 is an orifice for ejecting or dispensing a therapeutic agent 302, typically as a fluid. The nozzle may act as an atomizer to aerosolize the therapeutic agent 302, an aerator to generate a foam containing the therapeutic agent 302 or may simply spray or excrete the therapeutic agent 302. Nozzles 304 may be located at the tip of the therapeutic delivery device 308 or may be located on the sides of the device housing 126 of the therapeutic delivery device. The one or more nozzles 304 are in fluid communication with at least one therapeutic reservoir 306 containing at least one therapeutic agent 302. A nozzle 304 may protrude somewhat from surface of the therapeutic delivery device 308 or may be exposed by a sliding element or may be retractable, deployed upon positioning of the vaginal device 104 in the vaginal canal. The therapeutic agent 302 may be dispensed via the one or more nozzles 304 via a pressure discharge of the therapeutic reservoir 306 which may be occur due to an increase in pressure, a release of higher pressure in the therapeutic reservoir 306 than the surrounding space, or a reduction of volume in the therapeutic reservoir 306. The nozzle 304 may be designed to control the rate of release by limiting the flow of therapeutic agent 302. Similarly, the nozzle 304 may selectively direct the therapeutic agent 302 to a desired treatment site by directing the flow through one nozzle 304 or selectively directing the flow through one or more nozzles 304 while potentially keeping one or more of a plurality of additional nozzles 304 sealed.

A therapeutic reservoir 306 is a chamber containing at least one therapeutic agent 302. A vaginal device 104 or therapeutic delivery device 308 may contain one or more therapeutic reservoirs 306. The therapeutic reservoir 306 additionally comprises a means of discharging the contents of the therapeutic reservoir 306 via at least one nozzle 304. The discharge means may comprise a method of compressing the sides of the therapeutic reservoir 306 or a syringe like plunger mechanism designed to reduce the volume of the therapeutic reservoir 306 resulting in the therapeutic agent 302 within being discharged via one or more nozzles 304. The physical mechanism may comprise a spring or other device with stored potential energy which may be converted to kinetic energy when activated. In alternate embodiments, the therapeutic reservoir 306 may maintain an internal pressure which is greater than the atmospheric pressure outside the therapeutic delivery device 308 and vaginal device 104 when positioned in the vaginal canal thus allowing a small valve to be actuated, or a seal to be removed, allowing the contents of the therapeutic reservoir 306 to be discharged via one or more nozzles 304. Therapeutic delivery devices 308 is a device, method, or mechanism for delivering one or more therapeutic agents 302 to the mucosa of the vaginal canal.

A therapeutic delivery device 308 may comprise one or more mechanical delivery mechanisms 312 or automated delivery mechanisms 314. In some embodiments, a therapeutic delivery device 308 may be passive, such as holding a suppository in place or by allowing a therapeutic agent 302 on the outside of the therapeutic delivery device 308 to contact the mucosa of the vaginal canal or surrounding tissues, thus delivering the therapeutic agent 302. A therapeutic delivery device 308 may be an interchangeable component of a vaginal device 104 or may alternatively be integrated into a vaginal device 104. A therapeutic delivery device 308 may additionally comprise one or more of a nozzle 304, therapeutic reservoir 306, or dosing component 310.

A dosing component 310 is a component of a therapeutic delivery device 308 which controls the amount and/or rate of release of a therapeutic agent 302. In some embodiments, a dosing component may be internal to the therapeutic delivery deice 308, such as a dosing pump, or an actuator which can force an incremental amount of a therapeutic agent 302 from a therapeutic reservoir 306, such as in the case of a syringe like therapeutic reservoir 306 which may release the therapeutic agent 302 as the syringe plunger is compressed. In other embodiments, the dosing component 310 may comprise an amount of therapeutic agent which has been extruded onto the surface of the therapeutic delivery device 308, vaginal device 104, applicator 102, or therapeutic sheath 116. The dosing component 310 may additionally refer to a binder or carrier for the therapeutic agent which may delay, slow, or increase the rate of release of a therapeutic agent 302. In further embodiments, a dosing component 310 may refer to an external device which loads, fills, or applies one or more therapeutic agents 302 into or onto the therapeutic delivery device 308, vaginal device 104, applicator 102, or therapeutic sheath 116. The dosing component 310 may additionally be a component of a mechanical delivery mechanism 312 and/or an automated delivery mechanism 314.

A mechanical delivery mechanism 312 is a kinetic therapeutic delivery device 308. A mechanical delivery mechanism 312 may simply comprise a solid therapeutic agent 302, such as a suppository, which is held in place in the therapeutic delivery device 308 by an applicator 102 and which is released when the applicator 102 is removed after insertion of the vaginal device 104 into the vaginal canal. In further embodiments, a mechanical delivery mechanism may comprise one or more of a pump, actuator, spring, piston, servo, lever, screw thread, plunger, etc. or any other mechanism or device which can be automatically or manually activated such that activation of the mechanical delivery mechanism 312 delivers at least one therapeutic agent 302 to the mucosa of the vaginal canal. A mechanical delivery mechanism 312 may also be an automated delivery mechanism 314. In some embodiments, a mechanical delivery mechanism 312 may be activated manually, by pushing, twisting, pulling, sliding, etc. a part of the vaginal device 104, applicator 102, or a component of the vaginal device 104. In some embodiments, a mechanical delivery mechanism 312 may activate via an automated release or via a command from a mobile device 144.

An automated delivery mechanism 314 is a therapeutic delivery device 308 which activates in an automated or semiautonomous manner. Autonomous refers to not requiring direct manual actuation by a user. For example, a mechanical delivery mechanism 312 may automatically activate upon removal of an applicator 102 via normal insertion methods, without requiring a different action. In other embodiments, a mechanical delivery mechanism 312 may automatically release a therapeutic agent 302 after a dissolvable film or plug dissolves sufficiently to release the active ingredient 302 which may be held in a therapeutic reservoir 306 under pressure greater than the pressure in the vaginal canal or alternatively which may have another form of potential energy which is then released allowing the therapeutic agent 302 to be delivered. In further embodiments, an automated delivery mechanism 314 may receive a command from a mobile device 144. An automated delivery mechanism 314 may further control the dose and/or rate of release. In some embodiments, the does and/or rate of release may be dynamically controlled by a mobile device 144.

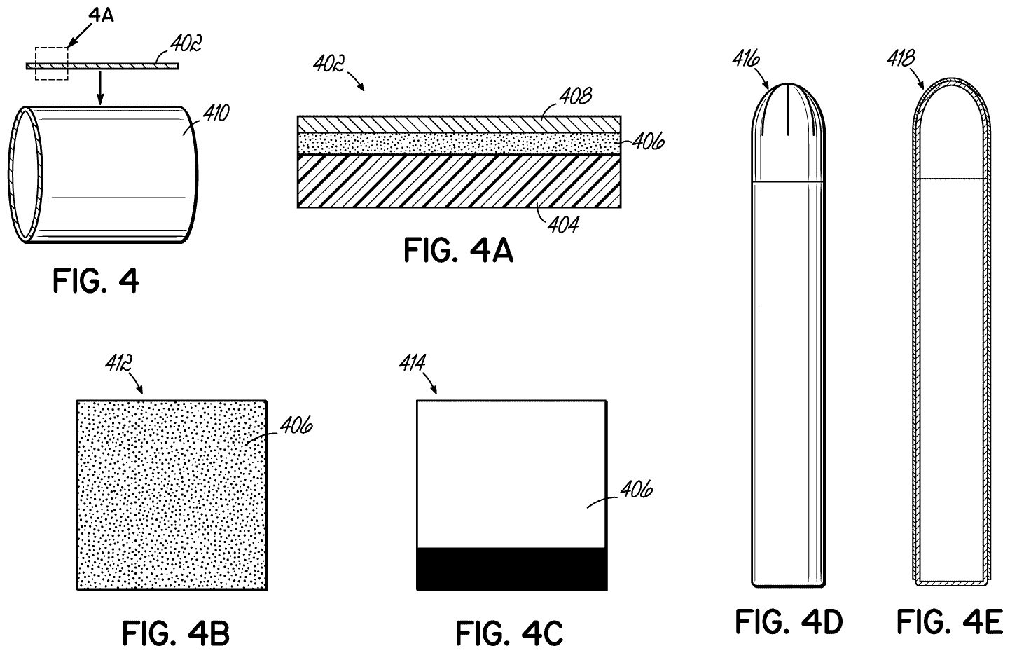

Functioning of the “therapeutic sheath” will now be explained with reference to FIG. 4. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

This figure displays the therapeutic sheath 116. The therapeutic sheath 116 comprises a therapeutic sheath 402 is constructed of an optional nonpermeable base layer sheath 404 which covers at least part of a vaginal device 418. The therapeutic sheath 402 may additionally comprise a therapeutic agent 406 deposited upon the sheath 404. The therapeutic agent 406 may further be covered by a water-soluble film 408 preventing the therapeutic agent 406 from immediately dissolving or being displaced during the insertion of the vaginal device 418 or while the vaginal device 418 is in the applicator 416. In some embodiments, the therapeutic sheath 402 may completely cover the vaginal device 418, similar to a condom, sized to the device and may be comprised of similar materials such as silicone, nitrile, latex, etc. In other embodiments, the therapeutic sheath 402 may only cover a portion of the vaginal device 418, such as the cylindrical shaft, while the tip of the vaginal device 418, an additional therapeutic delivery device 106, or one or more sensors 120, remain exposed.

The sheath 404 may comprise any material which is safe for contact with the mucosa of the vaginal wall and surrounding tissues. Exemplary embodiments are common materials used for condoms such as latex rubber, plastic, silicone, etc. The sheath 404 may range from 1 mil to 60 mils in thickness. In some embodiments, the sheath 404 may be easily cut or torn according to markings on the therapeutic sheath 402 indicating a dosage such that the dosage may be customized by sizing the therapeutic sheath 402 accordingly. In other embodiments, the sheath 404 may be oriented so as to direct a therapeutic agent 406 to a specific location. In some embodiments, a sheath 404 may be used without a therapeutic agent 406 to provide protection for the vaginal device 418 without delivering a therapeutic agent 406. This may be the case if a different therapeutic delivery device 106 is being utilized or the vaginal device 418 is being used to perform a diagnostic function. In some embodiments, the sheath 404 may be permeable, semipermeable, or comprised of a water-soluble film.

A therapeutic agent 406 is comprised of at least one active ingredient from the categories of pharmaceutical, medicinal, or holistic compositions. When deposited on a sheath 404 to form a therapeutic sheath 402, the therapeutic agent 406 is often in a cream or gel state. In other embodiments, the therapeutic agent 406 may be a fluid. In further embodiments, the therapeutic agent 406 may be a solid, particularly when mixed with a water-soluble material or binder. The water-soluble material may delay or slow the time or rate of release of the therapeutic agent 406. Additionally, in a solid form, a therapeutic agent 406 may be more precisely deposited on the sheath 404 allowing for precise dosing to specific regions within the vaginal canal. The therapeutic agent 406 may be enclosed within a water-soluble film 408 or may be directly exposed. In some embodiments the therapeutic agent 406 may be deposited on an applicator 416. In another embodiment the therapeutic agent 406 may be deposited on a vaginal device 418.

A water-soluble film 408 is comprised of a bio-safe material such as ARCare 93488 non-tacky dissolvable film made commercially available from a company called ARx. The water-soluble film 408 optionally protects a therapeutic agent 406 disposed on a therapeutic sheath 402 from being displaced or applied to the wrong location during insertion of a vaginal device 418. The water-soluble film 408 may additionally provide a delay in the release of the therapeutic agent 406 or may alternatively slow the rate of release of the therapeutic agent 406. In some embodiments, the water-soluble film 408 may dissolve at varying rates based on the thickness or composition of the water-soluble film allowing different timings, rates of release, or an ordered release of one or more therapeutic agents 406. For example, a first therapeutic agent 406 may be released to provide a numbing or pain relief function while a second therapeutic agent 406 may be released after the majority of the first therapeutic agent 406 is released such that the second therapeutic agent 406 comprises an antibacterial. In another embodiment, the water-soluble film 408 may allow a release of a therapeutic agent 406 such as ibuprofen at an initial rate of 5 mg per minute increasing to a rate of 10 mg per minute after the first 10 minutes to a maximum total dose of 200 mg. The water-soluble film 408 may be formulated such that the rate of release is proportional to the absorption rate of the therapeutic agent 406 being released.

A device housing 410 comprises structure to a vaginal device 418 and additionally provides protection for interior structures of the vaginal device 418. For example, the interior of a vaginal device 418 may be comprise of a plastic material. The plastic material may be covered by a sealed biocompatible material such as silicone preventing intrusion of fluids into the interior of the vaginal device 418. This may protect electronics within the vaginal device 418 from coming in contact with therapeutic agents 406, bodily fluids, or liquids used to clean the vaginal device 418. Likewise, the biocompatible material additionally protects the user from the internal components which may include materials that are not inherently biocompatible or otherwise safe for insertion into the human body. A therapeutic sheath 402 lays generally parallel to the exterior surface of the device housing 410. The sheath 404 may cover most of the surface of the device housing 410 or may alternatively cover only a portion of the device housing 410 such as no more than 50 percent. In some embodiments, a sheath 404 may cover the entirety of the device housing, however the therapeutic agent 406 may cover only a portion of the sheath 404.

A patterned therapeutic sheath 412 is a therapeutic sheath 402 where the therapeutic agent 406 is a structured material which is applied, printed, or deposited in a pattern over the surface of the sheath 404. The pattern may be regular, symmetrical, and may form a gradient. In some embodiments, the pattern may vary across the surface of the sheath 404 depending upon the area contacted by the sheath 404 and the intended treatment area. For example, a therapeutic agent 406 may utilize a more dense pattern near the tip of the device to facilitate an increase dose and rate of delivery, while a more dispersed pattern may be used at the base of the sheath 404 where less therapeutic agent 406 is needed. The pattern selected may comprise dots, lines, or other shapes. The pattern is largely dictated by the dosage required which impacts the density of therapeutic agent 406 applied, printed, extruded, or deposited, the rate of release which impacts the surface area of the therapeutic agent 406, and the duration of the release which impacts the depth of the pattern. In some embodiments, multiple therapeutic agents 406 may be interspersed within the pattern, such as alternating dots of two different therapeutic agents 406. In other embodiments, therapeutic agents 406 may be applied in one or more layers. The patterned therapeutic sheath 412 generally requires a solid therapeutic agent 406 such as one that has been mixed with a binder which is water soluble. The binder may be formulated to further impact rate of release. Likewise, the pattern, binder, etc. may be configured such that the rate of release is proportional to the absorption rate of the therapeutic agent 406 being released. In some embodiments, the patterned therapeutic agent 406 may be applied, printed, or deposited directly on the vaginal device 418 or applicator 416 instead of a therapeutic sheath 402.

A gradient sheath 414 is a therapeutic sheath 402 where the therapeutic agent 406 is a structured material which is applied, printed, extruded, or deposited in a gradient over the surface of the sheath 404. Like the patterned therapeutic sheath 412, the type and amount of therapeutic agent 406 may vary across the sheath 404. A gradient therapeutic sheath 414 may be comprised of solid therapeutic agents 406 or may utilize therapeutic agents in other forms such as a fluid, cream, gel, etc. The gradient may be constant or may vary depending on the desired treatment area. For example, the gradient may increase the concentration of therapeutic agent 406 on a surface intended to contact the front part of the vaginal canal and may diminish further away from the intended treatment site. In some embodiments, the gradient disposition of the therapeutic agent 406 may be applied, printed, extruded, or deposited directly on the vaginal device 104 or applicator 416 instead of a therapeutic sheath 402. In other embodiments, therapeutic agents 406 may be applied in one or more layers.

An applicator 416 may be used with a therapeutic sheath 402 or without a therapeutic sheath 402. Generally, when an applicator 416 is used with a therapeutic sheath 402, the therapeutic sheath 402 is being used to deliver a pretreatment therapeutic agent 406 prior to another therapeutic agent 406 being delivered by the vaginal device 418 or prior to a diagnostic function being performed by the vaginal device 418 though such a use is not required. A therapeutic agent 406 may alternatively be deposited directly on an applicator 416 to perform a similar purpose as using a therapeutic sheath 402. This would be particularly useful for provided a pretreatment delivery of a therapeutic agent 406 via the applicator 416 without having to remove and reinsert the applicator 416 to deploy the vaginal device 418. Alternatively, use of a therapeutic sheath 402 which wraps around the cylindrical shaft of the applicator 416 instead of fully covering the applicator 416 including the tip, would also facilitate a single insertion operation while effecting a pretreatment. Direct application of a therapeutic agent 406 to an applicator 416 may be desirable when the applicator 416 is disposable and intended for only a single use. When a therapeutic agent 406 is deposited directly on an applicator 416, it may be manually applied, automated with no discernable pattern to the application of the therapeutic agent 406, or the therapeutic may be applied similarly to the therapeutic agent 406 on a patterned therapeutic sheath 412 or a gradient therapeutic sheath 414.

A vaginal device 418 may be used with or without a therapeutic sheath 402. When a therapeutic sheath 402 is not used, a vaginal device 418 may or may not have a therapeutic agent 406 applied directly to the vaginal device 418. When a therapeutic agent 406 is deposited on the device housing 410 of a vaginal device 418, the therapeutic agent 406 may be used as a pretreatment, the primary treatment, or a secondary treatment. When a therapeutic agent 406 is deposited directly on the vaginal device 418, it may be manually applied, automated with no discernable pattern to the application of the therapeutic agent 406, or the therapeutic may be applied similarly to the therapeutic agent 406 on a patterned therapeutic sheath 412 or a gradient therapeutic sheath 414.

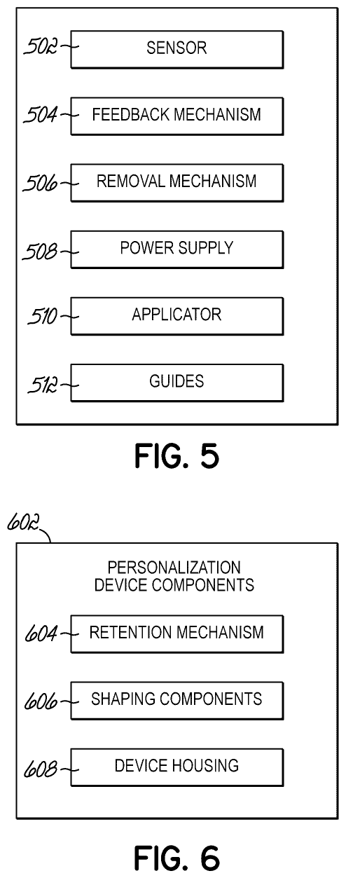

Functioning of the “feedback mechanism” will now be explained with reference to FIG. 5. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

This figure displays the feedback mechanism 130. The feedback mechanism 130 comprises a sensor 502 is used to measure one or more metrics, characteristics, or conditions, of a target subject. One of more sensors 502 may be integrated into either the applicator 102 or vaginal device 104, or both. Sensor 502 may be designed to detect any of a number of parameters, including but not limited to temperature, pH, the presence of a pathogen, detection of “healthy” microbiome bacteria, lubrication, etc. In one embodiment, a sensor 502 may be a digital device which may transmit data to any of communications system 142, including mobile device 144, cloud 146, and third-party network 148. Sensor 502 may further provide an indication that a treatment, or a step of a treatment is complete. In one example, sensor 502 is an electrical contact that is only made when therapeutic agent 108 is fully released, and then sends a signal to communications system 142 on the change in status.

A feedback mechanism 504 provides a signal, message, or any form of feedback to the user, based on a condition detected by sensor 502, and can be conveyed via lights, sounds, vibration, alerts, timer. Feedback mechanism 504 may be provided by vaginal device 104, or removal mechanism 506, or by communications system 142, including mobile device 144, cloud 146- and 3rd party network 148.

Feedback mechanism 504 can convey when a diagnostic function has been completed or a therapeutic agent 108 has been fully administered and/or absorbed which may be communicated to a user device 126 or may alternatively provide an indication via another means such as a vibration, electrical stimulation, etc. In one embodiment, sensor 502 detects that a desired level of lubrication or hydration is complete and sends a signal to mobile device 144 via Bluetooth. Feedback mechanism 504 may constitute a printed message on mobile device 144 (e.g., “Treatment is complete”), or alternatively may play a sound, create a vibration, or flash a light to indicate treatment has been completed. In another embodiment, sensor 502 detects a pathogen and sends a signal via Bluetooth to mobile device 144, which in turn is sent via cloud 146 to third party network 148, which may inform a healthcare professional more quickly of a developing problem to be treated.

A removal mechanism 506 is an element or feature allowing the vaginal device 104 to be easily removed from the vaginal canal. In the simplest embodiments, the removal mechanism 506 may be a string secured to the vaginal device 104. In some embodiments, removal mechanism 506 may additionally comprise a feedback mechanism 504. Examples of feedback mechanism may be a color indicator that treatment is complete, or that a condition has or has not been detected.

A power supply 508 is a device for supplying electricity to a vaginal device 104. A power supply 508 may be a single use or rechargeable battery. Alternatively, a power supply 508 may receive power wirelessly such as via wireless power transfer. An example of wireless power transfer utilizes inductance such as the Qi charging standard. In further embodiments, the power supply 508 may be a power cord connected to the vaginal device 104.

An applicator 510 is a device for delivering a vaginal device 104 to the treatment site in contact with the walls of the vaginal canal. In an embodiment, the applicator 510 is similar to a tampon applicator. In further embodiments, a traditional tampon applicator can be used to apply a vaginal device 104. The vaginal device 104 may be placed within the applicator 510, or may be prepackaged within an applicator 510, and can be inserted into the vaginal opening at which point the applicator 510 may be removed, leaving the vaginal device 104 in the vaginal canal in contact with the mucosa of the vaginal wall. In some embodiments, an applicator 510 may be functional in that is may also deliver therapeutic agents 108. For example, an applicator may have a therapeutic agent 108 which relieves pain or numbs the tissues which it contacts to provide a pretreatment or treatment when the applicator is inserted into the vaginal canal. A guide 512 comprises messaging, instructions, or information needed to lead a user through proper steps of using vaginal device 104.

Guide 512 may provide information needed prior to, during, or after treatment using vaginal device 104. In a first example guide 512 may be user instructions accessible to a user via QR code connected to an app for proper product use. A user may purchase a vaginal device 104 product, with a QR code printed on the packaging, within an included instructions booklet, or via website or email offer. Using mobile device 144, user may scan the QR code to allow direct access to online instructions or to download an app with instructions, or potentially multiple ways for feedback mechanism 504 to convey information. In another example, guide 512 can be standalone instructions, for example an automated checklist or questionnaire provided by a healthcare professional to select the proper vaginal device 104 for purchase. The digital questionnaire may be designed by a healthcare professional to determine whether a specific form of vaginal device 104 is appropriate for the situation, or whether immediate healthcare advice is suggested.

Functioning of the “personalization device components” will now be explained with reference to FIG. 6. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

This figure displays the personalization device components 134. Personalization device components 134 comprise personalization device components 602 may consist of specific ways to position vaginal device 104 properly or to customize the shape of vaginal device 104 to best suit a unique user. In some embodiments, personalization device components 602 comprise aspects of operation that allow for user choices. In one example, the user may elect to set specific timers or alerts for vaginal device 104 via mobile device 146. In another example, the user may select from a set of therapeutic or holistic agents to determine a personalized order of delivery, or selected dosage depending on the desired treatment or effect. In yet another example, the user may have choices to determine the best shape and fit of vaginal device 104 within a kit or set of products sold as a package.

The personalization device components may be further classified as retention mechanism 604 or shaping component 606. A retention device 604 is a mechanism or structure of a vaginal device 104 which may either hold the vaginal device 104 in a desired location within the vaginal canal or prevent the discharge of fluids, primarily to prevent therapeutic agents 108 from escaping the vaginal canal. In some embodiments, the retention device 604 is a shaped portion of vaginal device 104 that may be comprised of a flare, for example a donut shape, at the base of the vaginal device 104, or may alternatively comprise a wicking component at the base of the vaginal device 104 or a removable attachment to an external absorbent article, such as a nonwoven pad with an absorbent capability. The materials of choice for retention device 604 may be selected for flexibility and comfort, such as medical silicone, polymers, elastomers etc.

A shaping component 606 is a mechanism or structure of a vaginal device 104 which may either hold the vaginal device 104 in a desired location within the vaginal canal or prevent the discharge of fluids, primarily to prevent therapeutic agents 108 from escaping the vaginal canal. In one example, shaping component 606 is a deployable mechanism, such as a balloon which may be inflated upon insertion to provide a seal at the entrance of the vaginal canal. In another example, vaginal device 104 may contain an expandable sleeve, extended with a screw mechanism to adjust the length of vaginal device 104. In a further example shaping component 606 can further include a sensor 120 in communication with communications interface 122 so that data can be collected from shaping component 606 relevant to treatment, efficacy of delivery, best fit, etc. In other embodiments, shaping component 606 is pre-designed to suit a specific user, such as a smaller and/or narrower vaginal device 104 intended for users with smaller vaginal canals (e.g., younger women, pre-menstrual females, etc.).

A device housing 608 provides the structure and protection for the vaginal device 104 and its components. The device housing 608 comprises a bio-compatible material such as silicone, nitrile, etc. and fully encloses the components within the vaginal device 104 and/or therapeutic delivery device 106. In some embodiments, device housing 608 may be more rigid, and in other embodiments the material may be more flexible to help adjust to unique vaginal shape and contours. Device housing 608 may be constructed of a variety of materials, including medical silicone, polymers, elastomers etc.

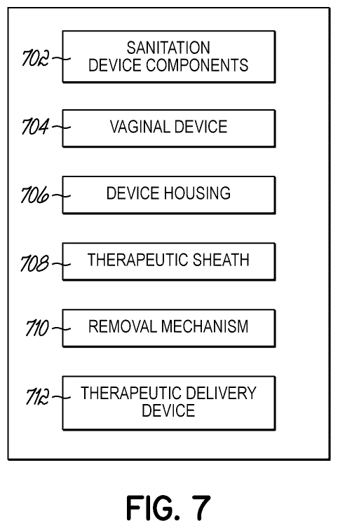

Functioning of the “sanitation device components” will now be explained with reference to FIG. 7. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

This figure displays the sanitation device 140. The sanitation device 140 comprises sanitation device components 702 include features, devices, objects, and assistance in disposing, reusing, or cleaning the various components of vaginal device 704. In some embodiments, vaginal device 704 is designed to be disposable, and in other embodiments, vaginal device 704 is designed to be used more than once. Sanitation device components 702 and their applications may vary, depending on whether vaginal device 704 and its components are disposable or durable. Sanitation device 702 may comprise a disposable bag when vaginal device 704 is disposable. In another example, sanitation device 702 may comprise disposable tools to grip, hold and transfer vaginal device 704 after use, or may also comprise disposable gloves, all of which may come as part of a kit or package provided with vaginal device 704. When vaginal device 704 is durable (i.e., multiple use), sanitation device 702 may further comprise assistive device, compositions, or tools to clean and disinfect vaginal device 704 for future use. Sanitization device component 702 may comprise a smaller home version of an autoclave, or a UV light sanitization housing, or a small tank to hold a cleansing solution such as an antibacterial agent, soap, alcohol etc. all designed to hold vaginal device 704, including device housing 706, removal mechanism 710 and therapeutic delivery device 712.

Activation methods may include steam, high temperature, soaking, agitation, vibration, or light application, all designed to clean remaining residue of therapeutic sheath 708 and sanitize vaginal device 704, including device housing 706, removal mechanism 710 and therapeutic delivery device 712 for future use. Sanitation device component 702 may further comprise tests, tools, or diagnostics to determine whether vaginal device 704 and other associated components are clean and ready for use.

A vaginal device 704 is comprised of non-absorbent materials and having a generally cylindrical shape, a curved and tapered leading end, and a cylindrical outer surface. The vaginal device 704 may additionally comprise a therapeutic delivery device 106 for administering at least one therapeutic agent 108 via the mucosa of the vaginal canal and may additionally comprise one or more of a device housing 120, a therapeutic sheath 116, sensors 120, communications interface 122, electronics power supply 124, retention mechanism 136, and a removal mechanism 132. The vaginal implant may fit in a traditional tampon applicator and be inserted into the vaginal canal following a similar procedure to applying a tampon. The construction of vaginal device 704 may be designed with either disposability or durability in mind. If disposable, vaginal device 704 may be constructed of lighter weight, less long-lasting materials. If durable, vaginal device 704 may be constructed of materials that are designed to allow for adequate cleaning for multiple uses. In one example, naturally antibiotic materials, such as silver, may be integrated into vaginal device 704, device housing 706, therapeutic sheath 708, removal mechanism 710 and therapeutic delivery device 712. In another example, medical grade silicone may be used as a component of construction for vaginal device 704, device housing 706, therapeutic sheath 708, removal mechanism 710 and therapeutic delivery device 712 to allow for more effective cleaning.

A device housing 706 provides the structure and protection for the vaginal device 704 and its components. The device housing 706 comprises a bio-compatible material such as silicone, nitrile, etc. and fully encloses the components within the vaginal device 104 and/or therapeutic delivery device 106. The device housing 706 may additionally provide the structure of the vaginal device 104 and/or therapeutic delivery device 106. In some embodiments, the device housing 706 may be comprised of an inner part which does not need to be constructed of bio-compatible materials and an outer portion designed to contact the body. Sanitation device component 702 may be designed to specifically clean and sanitize device housing 706.

A therapeutic sheath 708 is applied to at least a portion of the cylindrical outer surface of the vaginal device. The therapeutic sheath 708 may comprise a sheath of impermeable film material which completely envelops the vaginal device 704 or may instead only partially cover the vaginal device 704. In some embodiments, the therapeutic sheath 708 may be optionally used. The therapeutic sheath 708 may be at least partially coated in a therapeutic agent 108 and may additionally be enclosed within a water-soluble film to prevent the therapeutic agent 108 from being displaced during application of the vaginal device 704 or to delay the release of the therapeutic agent 108. In some embodiments, the therapeutic agent 108 may be combined with the water-soluble film or a similar composition to facilitate an extended release of the therapeutic agent 108. Sanitation device component 702 may be designed to specifically remove any remaining therapeutic agent 108 along with any undissolved traces of water-soluble film on therapeutic sheath 708. In some embodiments, the therapeutic sheath 708 may be disposable. Similarly, the therapeutic sheath 708 may function to isolate the vaginal device 704, limiting the need to sanitize the vaginal device 704 between uses, or making sanitation quicker and easier.

A removal mechanism 710 is an element or feature allowing the vaginal device 704 to be easily removed from the vaginal canal. In the simplest embodiments, the removal mechanism 710 may be a string secured to the vaginal device 704. In some embodiments, removal mechanism 710 comprises a sturdier design in order to support communication, control or power transfer for vaginal device 704. The sanitation device component 702 may be designed to specifically clean and sanitize removal mechanism 710, particularly where removal mechanism 710 is part of a multiple use design and more likely to have additional functions to support communication, control, or power transfer for vaginal device 704. In some embodiments, the removal mechanism 710 is disposable, and can be detached from the vaginal device 704 and replaced between uses. In other embodiments, removal mechanism 132 may be comprised of silicone or other polymeric materials, which may be cleaned and reused.

A therapeutic delivery device 712 is a component of a vaginal device 704 for delivery of a therapeutic agent 108, such as a pharmaceutical, medicinal, or holistic composition. A sanitation device component 702 may be designed to remove any remaining therapeutic agent 108 on, along, or within a therapeutic delivery device 712. This may comprise purging a reservoir or other storage apparatus for containing a therapeutic agent. Additionally, a therapeutic delivery device 712 may comprise an interchangeable module which can be swapped between uses. A therapeutic delivery device 712 may additionally be disposable, intending to be used once and discarded, recycled, or otherwise disposed of. Therapeutic delivery devices 712 may be refurbished and recharged with a therapeutic agent 108 to facilitate reuse. Such refurbishment could operate as part of a subscription service.

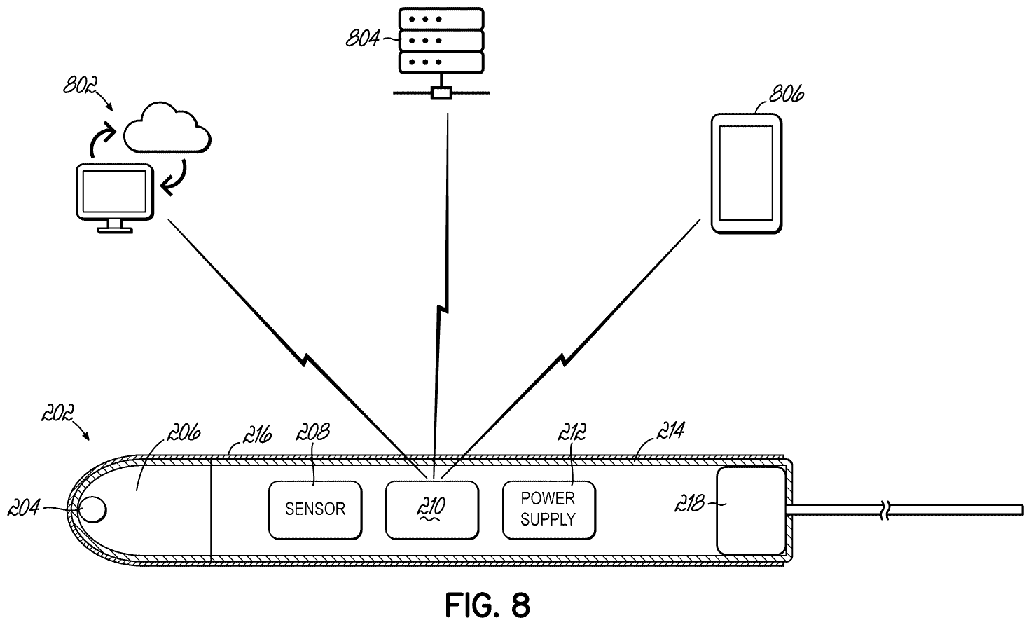

Functioning of the “communications system” will now be explained with reference to FIG. 8. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

This figure displays the communications system 142. The communications system 142 comprises a cloud 802 is a distributed network of computational or data storage resources. In some embodiments, a cloud 802 may additionally be secured via encryption. A cloud 802 may communicate with a vaginal device 808. In some embodiments, the vaginal device 808 may connect directly to the internet via a communications interface 810 or may connect to a cloud 802 indirectly via a connected device such as a mobile device 806. The cloud 802 may house data collected by one or more vaginal devices 808 or may run one or more applications for use with a vaginal device 808 with or without a mobile device 806. A cloud 802 may also enable the communication of data from a vaginal device 808 to a remove mobile device 806 such as a mobile device 806 in possession of a medical professional. A 3rd party network 802 is a proprietary network comprised of private resources which may be made available based upon registration and/or agreement to the private party's terms of service. In an embodiment, a 3rd party network 804 may comprise one or more servers, databases, application, etc. operated by a 3rd party to provide additional functionality to a vaginal device 808, such as tracking data measured by one or more sensors 812, tracking activity such as how often a therapeutic agent 108 was used, the dosage, and the reason for treatment. In some embodiments, a 3rd party network 804 may be a cloud 802 resource provider such as Microsoft Azure, Amazon Web Services, etc. used to host one or more applications.

A mobile device 806 is a computing device which may be used by the user of a vaginal device 104 or alternatively by a medical professional to receive data from a vaginal device 808 via a communications interface 810. The mobile device 806 may be any of a cell phone, tablet, computer, or other proprietary device. In some embodiments, the mobile device 806 may receive data by recording audio from the user or sounds emitted by the vaginal device 808. In other embodiments, the mobile device 806 may receive data via a camera, such as taking a picture or video of a pH strip. An app on the mobile device 806 may be used to interpret the image, such as to determine the pH measured by the vaginal device 808.

In other embodiments, the image taken may be of an assay or the therapeutic delivery device 106 to verify that the therapeutic agent 108 was properly applied. The mobile device 806 may receive a notification, alert, or instruction from the vaginal device 808 when it has completed its intended function and is ready to be removed. In some embodiments, the mobile device 806 may function as a timer, such that the user indicates when the vaginal device 808 was inserted and the mobile device 806 provides a notification when a suitable amount of time has elapsed and the vaginal device 808 should be removed. The mobile device 806 may additionally allow for control of the vaginal device 808, such as user configurable personalization settings including controlling shaping components 138 and may additionally allow for control of a dose and rate of release of a therapeutic agent 108.

A vaginal device 808 comprises a therapeutic delivery device 106 and may additionally comprise a communications interface 810, and one or more sensors 812. Data collected by one or more sensors 812 may be relayed to one or more of a mobile device 806, cloud 802, or 3rd party network 804 via a communications interface 810. The vaginal device 808 may receive configuration data including personalization settings and configuration data related to one or more therapeutic agents 108 which may be released via one or more automated delivery mechanisms 114 via the communications interface 810. The vaginal device 808 may be paired to a single mobile device 806 or the user may authorize the vaginal device 808 to transmit and share data to more than one mobile device 806 directly or via the user's mobile device 806. In some embodiments the data collected from the vaginal device 808 may be stored in the cloud 802 or may alternatively be stored on a 3rd party network 804.

A communications interface 810 enables communication of a vaginal device 808 with one or more mobile devices 806, 3rd party networks 804, or a cloud 802. A communications interface 810 may use one or more wireless protocols such as Wi-Fi, Bluetooth, LTE, 4G, 5G, near field communication, etc. to communicate with a mobile device 806, 3rd party network 804, or cloud 802. In some embodiments, a communications interface 810 may be a physical interface such as an ethernet or USB port. A communications interface 810 may receive power from an electronics power supply 124 and may be in electrical communication with one or more sensors 812 on the vaginal device 808. The sensors 812 may be modular enabling different sensors 812 to be interchanged on the same vaginal device 808. A sensor 812 is a device for measuring one or more metrics or characteristics of a target subject. One or more sensors 812 may be integrated into the vaginal device 808 to measure or monitor one or more metrics such as temperature, pH, or the presence of a pathogen. In some embodiments, a sensor 812 may be a chemical indicator such as a pH strip which changes color when in the presence of fluids. In other embodiments, a sensor 812 may be a digital device which may transmit data to a connected mobile device 806 via a communications interface 810. The collected data may further be transmitted to a cloud 802 or 3rd party network 804. In further embodiments, a sensor 812 may be an assay. In some embodiments, sensors 812 may be modular, such as being. In some embodiments, a sensor 812 may detect when a diagnostic function has been completed or a therapeutic agent 108 has been fully administered and/or absorbed which may be communicated to a mobile device 806.

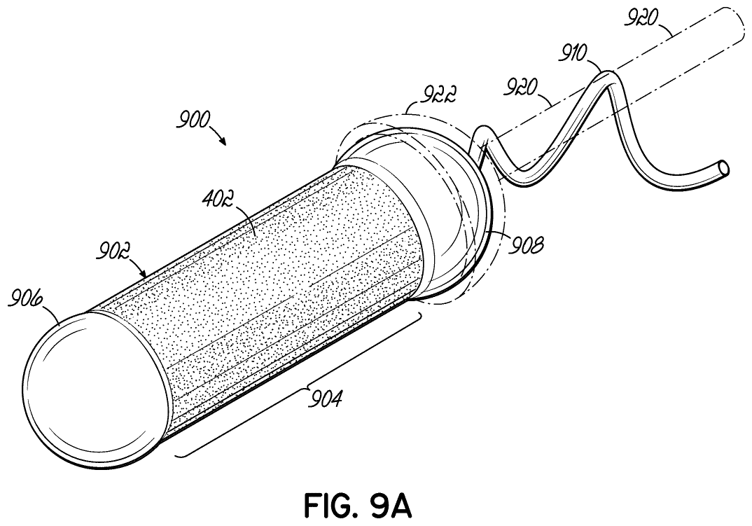

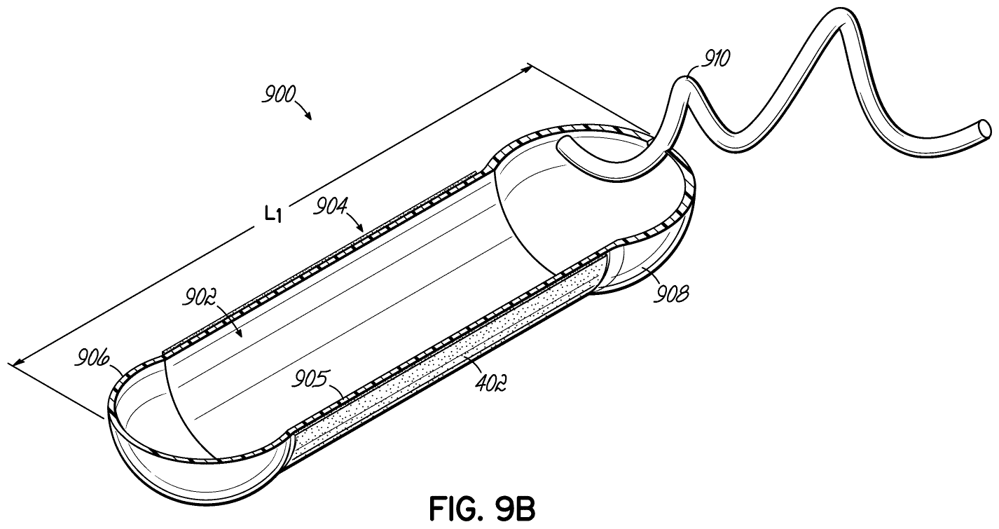

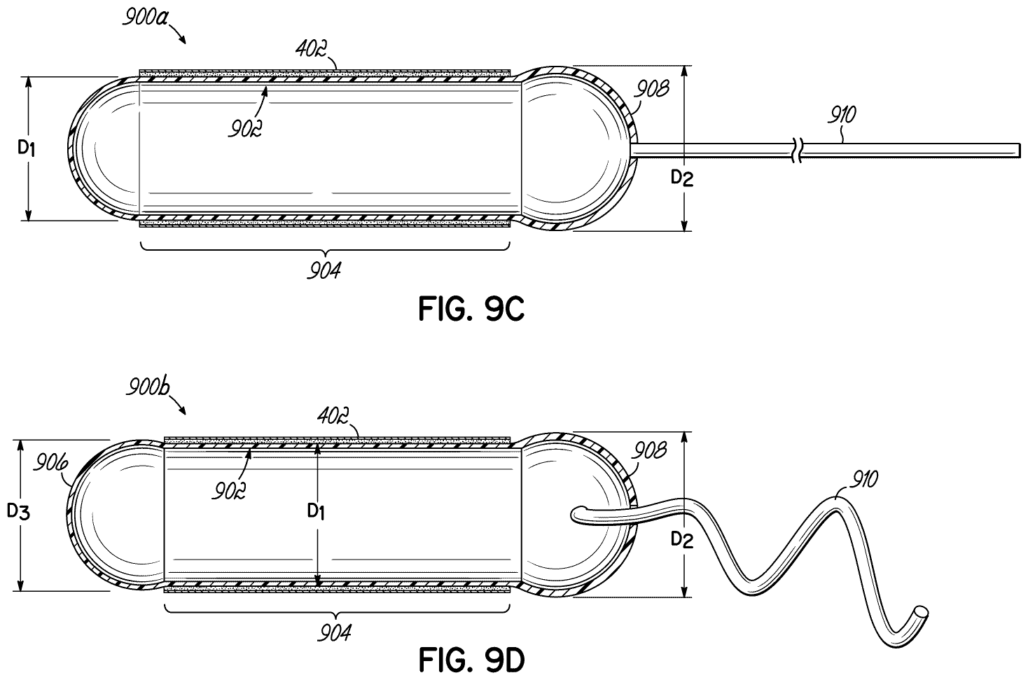

FIGS. 9A-9D illustrate one particular embodiment of a vaginal device in accordance with various features of the invention as described herein. FIG. 9A is a perspective view of a vaginal device or insert device 900 in accordance with one embodiment of the invention. Vaginal device 900 includes a body 902 (see FIG. 9B) that is generally tubular or cylindrical in shape. The body 902 may be generally hollow and tubular, as shown in various illustrated embodiments. Alternatively, it might be a more solid cylindrical body, such as formed by a foam. Generally, the body is formed of a suitable bio-safe and non-absorbent material which is safe for contact with the mucosa of the vaginal wall and surrounding tissues. Exemplary embodiments are common materials used for condoms such as latex rubber, plastic, silicone, or nitrile or a bio-safe and non-absorbent foam might be used. The body is preferably non-permeable and non-absorbent. In some of the disclosed embodiments, the generally hollow body might be formed by appropriate injection molding or blow molding techniques for providing a comfortable and flexible device that can be positioned and retained in the appropriate position and area for delivery of the desired therapeutic agent(s) 108 for the desired condition or disease that is address by the device 900. Device 900 is configured and dimensioned for insertion into the vaginal canal to contact the mucosal membrane of the vaginal canal.

To that end, the body 902 has an elongated form with opposing ends. Portions or sections of the body provide its functionality of holding one or more therapeutic agents 108 and positioning the one or more therapeutic agents 108 for delivery. Furthermore, the body has one or more sections for positioning and retaining the device in the desired location during delivery. Body 902 is elongated and includes a delivery section/portion or application section 904 between the opposing ends, which is a non-permeable section to act as the housing 410 for receiving therapeutic agents such as on a therapeutic sheath 402 as disclosed herein with respect to FIG. 4. As noted herein, one example of a sheath used in device 900 and other devices discussed herein may be at least partially coated in one or more therapeutic agent(s) 108 and may additionally be enclosed within a water-soluble film to prevent the therapeutic agent 108 from being displaced during application of the vaginal device 104 or to delay the release of the therapeutic agent 108. The delivery section 904 may receive, for example, a water soluble film or printed pattern containing therapeutic agent(s), drug or lifestyle components comprising up to 50% of the layer. Alternatively, a metalized foil(s) can be presented in the delivery section for therapeutic benefits or therapeutic process enablement. The device 900 may be utilized over a range of hours since there is no absorbent media of the device. The device may be used to simultaneously deliver multiple therapeutics. In the illustrated embodiment, the delivery section 904 of the body is cylindrical and has a cross-sectional diameter. Referring to the embodiment of FIG. 9B, the delivery section 904 could be formed by the thin flexible wall 905 of a molded body 902. The wall is flexible for some flexing and compression of the body. The delivery section 904 of the body wall may provide the base for holding one or more therapeutic agents or could be a base layer for a sheath that be applied or adhered to the delivery section, as disclosed herein with respect to FIG. 4. The elongated tubular body may be molded or otherwise fabricated in a shape for receiving a sheath of therapeutic agent(s). In the illustrated embodiment of FIGS. 9A-9B the body 902 forms a cylindrical tube having a generally circular cross-section and dimension D₁, such as a diameter dimension, as shown in FIG. 9C-9D.

The vaginal device 900 includes at least one section that has a larger dimension than the body dimension or the dimension of the delivery section. For example, one or more position stability or retention sections 906, 908, might have a dimension D₂ or D₃, such as a diameter dimension, that exceeds the dimension D₁ of the body delivery section. In some embodiments, the elements or sections 906, 908 are generally hemispherical sections. The larger position stability sections 906, 908 may be positioned at the ends of the body and act as retention elements or positioning and stability elements to engage the vaginal wall more firmly than the delivery section 904 and thereby secure, position and retain the device 900 and the delivery section in the proper position in the vaginal cavity for use. In the embodiment disclosed in FIGS. 9A-9B, the device 900 includes a plurality of larger sections 906, 908 for stabilizing the device 900 at two contact points against a vaginal wall. Those position stability or retention sections 906, 908 are linearly separated along the length of the body 902 of the device and positioned at the ends of the body. However, other similar such retention or positioning sections might be positioned elsewhere on the length of the body between the ends, such as in the delivery section 904.

In accordance with a further feature of the device 900, one of the position stability sections might be larger than the others for providing a section that acts as a device retention element or at least a primary retention element. Referring to FIG. 9D, the position stability section 908 has a dimension D₂ that exceeds a dimension D₃ of position stability section 906. In that way, section 908 engages the vaginal wall more forcefully and thus effectively acts as a retention element 908 that anchors the device 900 in position with respect to the vaginal wall and holds the device 900 in place for an amount of time for delivery of one or more desired therapeutic agent(s) 108. Positioned at either end, the elements 906, 908 provide stability in the vaginal cavity. Both of the positioning section/element 906 and retention section/element 908 have dimensions D₂ and D₃ that exceed dimension D₁ of the tubular delivery section and therefore, when the device 900 is inserted into the vaginal cavity, the delivery section 904 provides a recessed cylindrical surface for contacting the vaginal mucosal membrane and delivering the one or more therapeutic agents 108 from the sheath 402 in delivery section 904. In the illustrated embodiments, the elements 906, 908 are positioned generally at the opposing ends of the body.

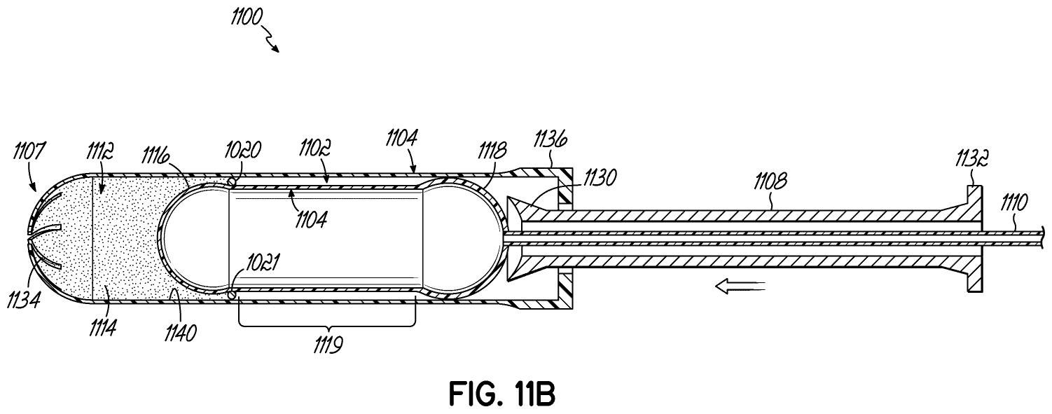

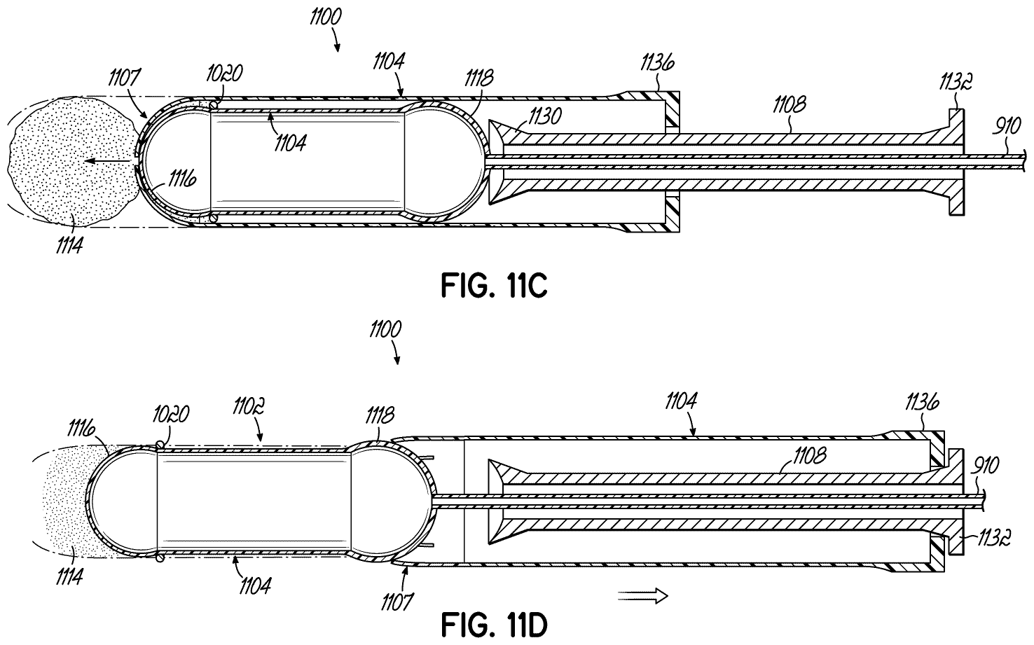

The sections 906, 908 cooperate with the length of the device body and the recessed cylindrical delivery section to provide bi-directional insert stability while maintaining the delivery section 904 in the desired position to increase therapeutic efficacy. The unique vaginal device maximizes tissue contact area for the delivery section and sheath and maximizes position stability. Depending on the size needed and the user, the length L₁ of the device body 902 might be in the range of 50-85 mm. A device body length in the range of 50-65 mm may be optimal for a device that goes inside of an applicator or application system as shown in FIG. 11A. Alternatively, different sizes might be offered in the length of the device. For example, a small size, medium size and large size the range of 50-85 mm might be offered. (The diameters D₁, D₂ and D₃ might be in the range of 7.5-17.5 mm, with the noted size differentials of the sections 904, 906, 908 implemented as appropriate. Generally, a device outside diameter in the range of 9 mm-20 mm would be suitable. The body 902, if hollow, would have walls with a thin dimension that would keep the device at a minimum weight while providing structural integrity.

The position stability section 906 and retention section 908, in one embodiment, have a greater rigidity than the delivery section 904 for securing and positioning the device while allowing flexibility of the delivery section 904 against the vaginal wall. The body is made of a non-absorbent material, such as a suitable plastic, and the outer surface thereof has a low coefficient of friction. The vaginal device is light weight for comfort with a low mass. The delivery area between the position stability section 906 and retention section 908 is horizontally pliable and bendable for compression. To that end, the position stability section 906 and retention section 908 may be molded to have a wall that has a greater thickness 914 than the thickness of wall 905 of the delivery section 904 (see FIG. 9B). For comfort in inserting the device 900 as well as the continued retention thereof, the ends of the body 902, in the form of the position stability section 906 and retention section 908, are smooth and generally spherical or hemispherical in shape. More specifically, as illustrated, the position stability section 906 and retention section 908 have hemispherical shapes that are radiused and transition to the lesser diameter of the cylindrical delivery section 904. The position stability section 906 and retention section 908 may be appropriately molded with the delivery section or otherwise secured with the ends of the cylindrical delivery section 904. Generally, for insertion, the front end of the vaginal device includes the position stability section 906, and its smooth and hemispherical shape ensures comfortable insertion. The trailing end of the vaginal insert device includes the retention section/element 908, and its smooth and hemispherical shape also ensures comfortable retention and positioning in the vaginal cavity. The device 900 thus provides multiple radially oriented anchor points. Of course, in an alternative embodiment, the retention section might be placed on the front end of insertion end of the device 900.

The vaginal device 900 is inserted into the vaginal cavity and positioned as desired. To that end, the device may be manually inserted or, alternatively, an elongated applicator 920 may be used. The applicator may have an end 922 that is configured to snap or otherwise be secured on the retention section 908 of device 900. The elongated applicator 920 may be used to push the device 900 into the desired position and then disengaged to leave vaginal device 900 in place. The unique shape and construction of vaginal device 900 ensures that it stays in position during an upright and active disposition of the wearer. Furthermore, the unique construction of the retention section ensures that the vaginal cavity is effectively plugged while in use so that any of the one or more therapeutic agents 108 delivered do not leak out while the user is upright. For removal, the vaginal device includes one or more retrieval or removal mechanisms 910, such as a string or cord that may be grasped to pull the device 900 out after usage. The smooth and hemispherical shape of the sections/elements 906, 908 also ensures comfortable removal of the device 900. The removal mechanism is preferably a non-absorbent material such as silicon.

As discussed herein, a therapeutic agent may be applied directly to the body of the device, such as at or on the delivery section or a therapeutic sheath 402 may be applied and/or secured cover some portion of the body such as at or on the delivery section 904 (see FIG. 4). In one embodiment the therapeutic agent or therapeutic sheath 402 may cover all or most of the delivery section 904. The sheath 404 may range from 1 mil to 60 mils in thickness. In some embodiments, a sheath 404 may be easily cut or torn according to markings on the therapeutic sheath 404 indicating a dosage such that the dosage may be customized by sizing the therapeutic sheath 404 accordingly for application or adhesion to the delivery section 904 of the device body. In other embodiments, the sheath 404 may be oriented or dimensioned on the delivery section 904 so as to direct a therapeutic agent 406 to a specific location.

A therapeutic agent 406 is comprised of at least one active ingredient from the categories of pharmaceutical, medicinal, or holistic compositions as disclosed herein. When deposited on the delivery section 904 of device 900 or on another substrate to form a therapeutic sheath 402 that is then applied to device 900, the therapeutic agent 406 is often in a cream or gel state. In other embodiments, the therapeutic agent 406 may be a fluid. In further embodiments, the therapeutic agent 406 may be a solid, particularly when mixed with a water-soluble material or binder. The water-soluble material may delay or slow the time or rate of release of the therapeutic agent 406. Additionally, in a solid form, a therapeutic agent 406 may be more precisely deposited directly on the device 900 or a sheath 404 that applied to or secured to the device 900 allowing for precise dosing to specific regions within the vaginal canal. The therapeutic agent 406 may be enclosed within a water-soluble film 408 or may be directly exposed for contact.

The vaginal device 900 is inserted and then generally left in place for an amount of time for the therapeutic agent to take effect. In that regard, in one embodiment of the invention, the device is a static device. In other embodiments, as described herein, the vaginal device 900 or a similar such device is part of a more dynamic system for delivering a dosage of a therapeutic agent and then remaining in place for an amount of time for the therapeutic agent to take effect or be absorbed.

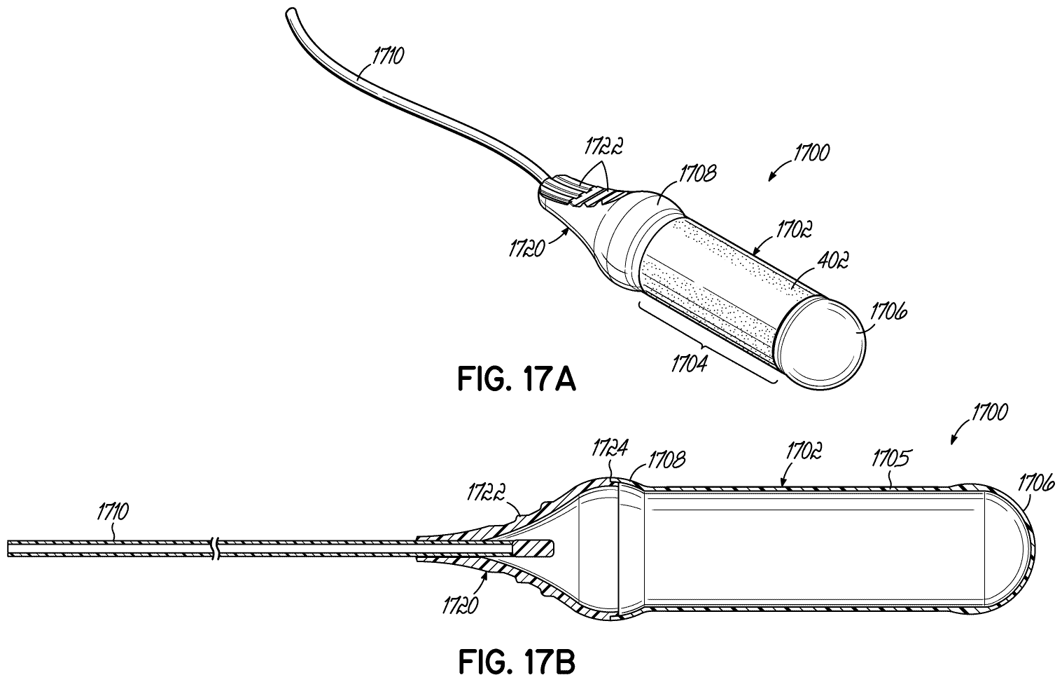

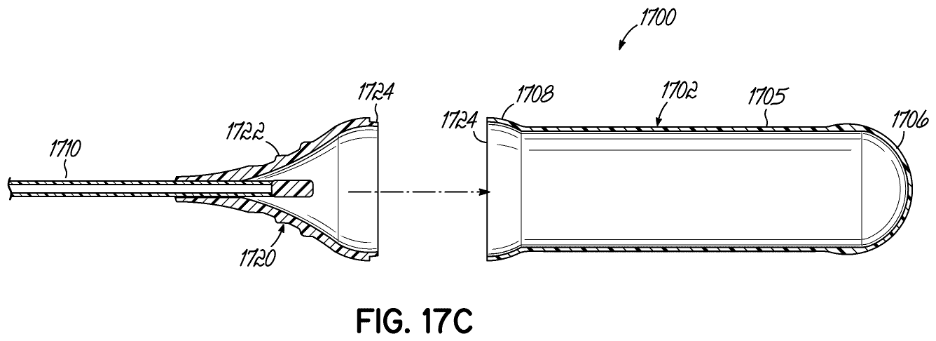

FIGS. 17A-17C illustrate another embodiment of a static vaginal device in accordance with the invention as described herein. FIG. 17A is a perspective view of a vaginal device or insert device 1700 in accordance with one embodiment of the invention. Vaginal device 1700 includes a body 1702 (see FIG. 17B) that is generally tubular or cylindrical in shape. The body 1702 may be generally hollow and tubular or it might be a more solid cylindrical body as discussed herein. It may be formed of similar materials as device 900 for example. Device 1700 is configured and dimensioned for insertion into the vaginal canal to contact the mucosal membrane of the vaginal canal.

To that end, the body 1702 has an elongated form with opposing ends. Portions or sections of the body provide its functionality of holding one or more therapeutic agents 108 and positioning the one or more therapeutic agents 108 for delivery. Furthermore, the body has one or more sections for positioning and retaining the device in the desired location during delivery. Body 1702 is elongated and includes a delivery section/portion 1704 between the opposing ends, which is a non-permeable section to act as the housing for receiving therapeutic agents such as on a therapeutic sheath 402 as disclosed herein. The delivery section 1704 of the body is cylindrical and has a cross-sectional diameter. Referring to the embodiment of FIG. 17B, the delivery section 1704 could be formed by the thin flexible wall 1705 of a molded body 1702. The elongated tubular body may be molded or otherwise fabricated in a shape for receiving a sheath of therapeutic agent(s). In the illustrated embodiment of FIG. 17A the body 902 forms a cylindrical tube having a generally circular cross-section and dimension as discussed herein.

The vaginal device 1700 includes at least one section that has a larger dimension than the body dimension or the dimension of the delivery section in the form of the position stability or retention sections 1706, 1708, with a diameter dimension that exceeds the diameter dimension of the body delivery section. The larger position stability sections 1706, 1708 may be positioned at the ends of the body and act as retention elements or positioning and stability elements to engage the vaginal wall more firmly than the delivery section and thereby secure and position the device and the delivery section in the proper position in the vaginal cavity for use. In the embodiment disclosed in FIGS. 17A, 17B, the device 1700 includes a plurality of larger sections 1706, 1708 for stabilizing the device at two contact points against a vaginal wall. Those position stability or retention sections are linearly separated along the length of the body of the device and positioned at the ends of the body as discussed herein. In accordance with a further feature of the device 1700, one of the position stability sections might be larger than the others for providing a section that acts as a device retention element or at least a primary retention element. Referring to FIG. 17B, the position stability section 1708 has a diameter dimension that exceeds a diameter dimension of position stability section 1706 for anchoring and retaining and positioning the device in position with respect to the vaginal wall and holds the device in place for an amount of time for delivery of one or more desired therapeutic agent(s) 108. Positioned at either end, the elements 906, 908 provide stability in the vaginal cavity.

The sections 1706, 1708 cooperate with the length of the device body and the recessed cylindrical delivery section to provide bi-directional insert stability while maintaining the delivery section 1704 in the desired position to increase therapeutic efficacy. The size and dimensions of the device 1700 may resemble those sizes and dimensions discussed herein with respect to device 900. Also, the position stability and retention sections have a greater rigidity than the delivery section for securing and positioning the device while allowing flexibility of the delivery section against the vaginal wall.

The body may be made of a non-absorbent material, such as a suitable plastic, and the outer surface thereof has a low coefficient of friction. The vaginal device is light weight for comfort with a low mass. The delivery area between the position stability section and retention section is horizontally pliable and bendable for compression. To that end, the position stability section 1706 and retention section 1708 may be molded to have a wall that has a greater thickness than the thickness of wall 1705 of the delivery section 1704 (see FIG. 9B). For comfort in inserting the device 1700 as well as the continued retention thereof, the ends of the body 1702, in the form of the position stability section 1706 and retention section 1708, are smooth and generally spherical in shape. More specifically, as illustrated, the position stability section and retention section have hemispherical shapes that are radiused and transition to the lesser diameter of the cylindrical delivery section.

The embodiment of FIGS. 17A-17C incorporates a manual feature for assisting in the positioning and placement of device 1700. Specifically, one end of the device, for example, the end proximate the retention section 1708, incorporates a shackle portion or grip portion 1720 that provides for a grip by a user for the insertion process. The grip portion 1720, might have ribs 1722, for example, to assist in a secure grip. For insertion, the front end of the vaginal dev

ice includes the position stability section 1706, and its smooth and hemispherical shape ensures comfortable insertion. The grip portion 1722 may be gripped by a user and used to manually push the device. The retention section/element 1708 has a smooth and hemispherical shape to ensure comfortable retention and positioning in the vaginal cavity. For removal, the vaginal device includes one or more retrieval or removal mechanisms 1710, such as a string or cord that may be grasped to pull the device 1700 out after usage. The smooth and hemispherical shape of the sections/elements 1706, 1708 also ensures comfortable removal of the device 1700.

Referring to FIG. 17C, in the manufacture of device 1700, the main portion of the body 1702 might be molded, such as by injection molding, and the grip portion might be separately molded and then secured to form the end of the device. For example, a portion of the hemispherical shape forming retention section/element 1708 might be molded as part of the body 1702, while another portion of the hemispherical shape might be molded with the grip portion 1722. The retrieval or removal mechanism 1710 might be secured to the grip portion 1722. Each of the separate portions might incorporate transition sections 1724 that facilitate the portions coming together to form the retention section 1708 with a laser weld or other weld around the seam of the transition sections for securing the portions together.

The vaginal devices 900, 1700 as illustrated provide a reshaping of the lower vaginal channel in preparation for the contraction and predictable mucosal tissue contact with the delivery section and the one or more therapeutic agents or a sheath. The devices can be used to target the vaginal vascular field. The inventive vaginal device provides a comfortable, secure and position stable therapeutic delivery process that enables normal physical activity. The proper positioning and retention ensure effective and complete delivery of the one or more therapeutic agents theoron. Furthermore, as discussed herein, multiple therapeutic agents may be delivered in concurrent or sequential patterns.

While some of the illustrated embodiments show a plurality of position stability/retention sections 906, 908, that act as elements to stabilize and retain the device 900, alternative embodiments might only implement a single such section or element. For example, referring to FIG. 9C, the vaginal device 900a has all the features of the device 900 as described herein. However, only a single position stability/retention section/element 908 is used to position and retain the device 900a in the vaginal cavity so that the delivery section 904 can dispense the therapeutic agent(s). The end 907 of the device 900a has the similar diameter or dimension as the delivery section 904 and is rounded or radiused for easy insertion. In still another embodiment, as illustrated by device 900b in FIG. 9D, the position stability/retention sections/elements 906, 908 might be similarly dimensioned (D₂=D₃), such as in diameter, to provide the desired position stability and also the retention that is needed.

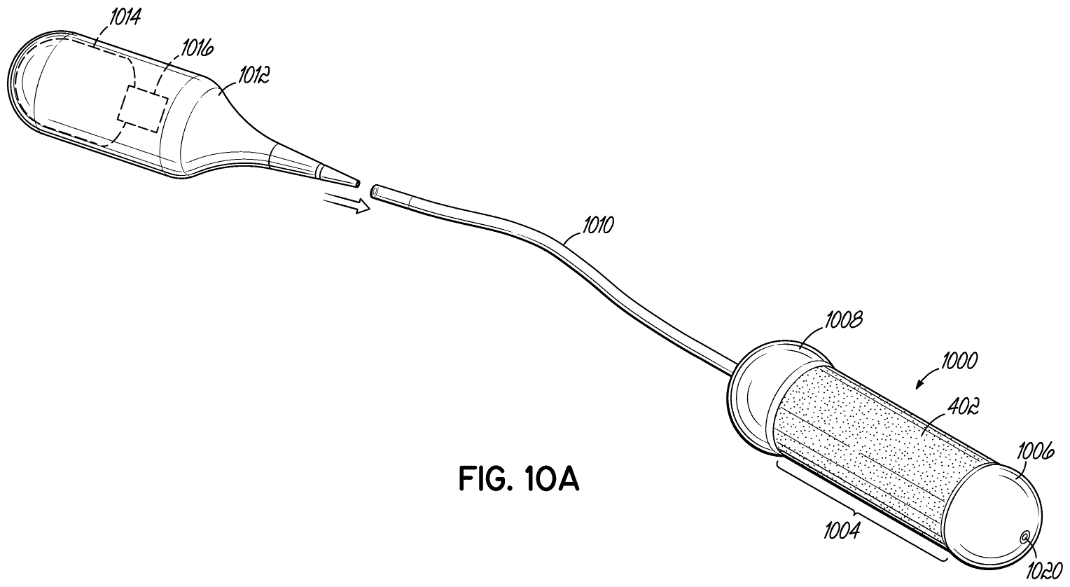

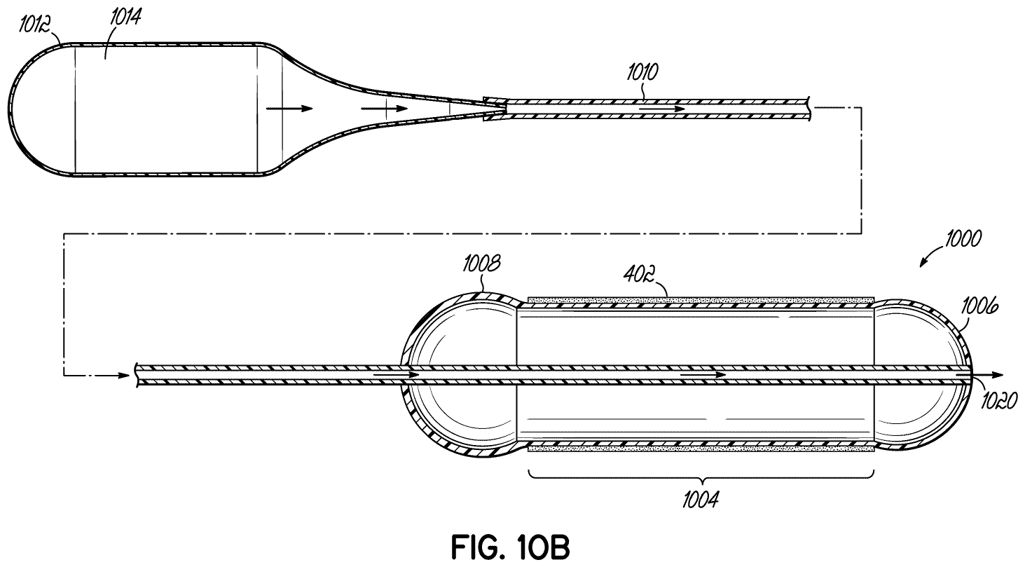

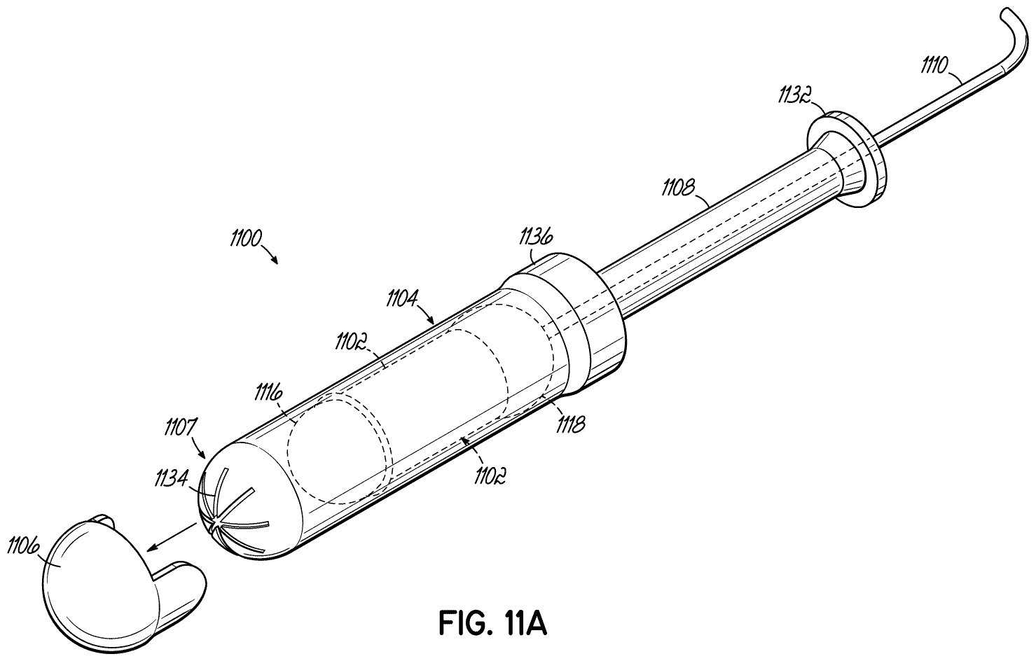

FIGS. 10A-10B illustrates an alternative embodiment of the invention that is able to be used for targeted delivery of one or more therapeutic agents as described herein. Specifically, the vaginal device 1000 includes a delivery system that is coupled with a supply or supply system that can selectively, dynamically and accurately deliver therapeutic agents to the vaginal cavity. That targeted delivery might be the primary delivery or alternatively, it can be delivered in combination with a delivery as discussed with device 900. The vaginal device 1000 generally resembles the vaginal device 900 as described herein in construction, dimension and operation and may be utilized in a similar fashion as device 900. That is, device 1000 may be inserted and held in position by one or more position stability/retention sections/element 1006 and 1008 to maintain a delivery section 1004 in the proper position to deliver one or more therapeutic agents as described. However, vaginal device 1000 also includes a delivery tube or capillary structure 1010 that is in communication with both the device 1000 and a supply system or supply and delivery system 1012 of therapeutic agent(s). That supply system 1012 may include one or more reservoirs or supplies 1014 of the therapeutic agent(s) and may also include a mechanized or manual device 1016 that couples with the reservoir 1014 to deliver the therapeutic agent(s) to the delivery tube 1010. To that end, the mechanized or manual device 1016 might include a pipette, a pump, a syringe, or may take the form of a flexible package or reservoir that might be manually squeezed by the user to deliver therapeutic agent(s) from a reservoir 1014 to the vaginal device 1000.

To that end, the vaginal device has at least one opening or orifice that is in communication with the delivery tube 1010 and system 1012 for dispensing a therapeutic agent. In the illustrated embodiment, the vaginal device 1000 includes orifice 1020 that communicates with delivery tube 1010. The delivery tube may extend through the hollow body of vaginal device 1000 as illustrated in the embodiment of FIG. 10B to couple to or communicate with the orifice 1020. The orifice 1020 might be formed in the forward or upper positioning/retention section or element 1006. As discussed, the therapeutic agent(s) 108 of reservoir 1014 may take any form as indicated by the most efficient or effective delivery method such as a fluid, a foam, a cream, a vapor, gas, a gel, mixtures or combinations of states of matter that may be delivered through tube 1010 to orifice 1020. The vaginal device 1000 of FIGS. 10A-10B provides a targeted delivery of therapeutic agent(s) 108 to an area of the vaginal canal, above where the device 1000 is located and positioned, such as to the uppermost vaginal vascular field or cervical or uterine vascular field or area.

The targeted delivery of the therapeutic agent(s) 108 may be operated as the primary path of delivery through the device 1000. In such a case, the device 1000 that is positioned in the vaginal cavity can be removed after application of the therapeutic agent(s) 108, or alternatively, it can remain in place and act as a physical plug to contain the delivered therapeutic agent(s) 108 when the user is upright. In such usage, the delivery section 1004 of the device might be empty or not contain any therapeutic agent(s) 108. That is section 1004 would just be smooth material.

Alternatively, the device 1000 may also provide an additional or synergistic delivery of one or more therapeutic agent(s) 108 through the delivery section 1004 in conjunction with the primary delivery through orifice 1020. To that end, the vaginal device might incorporate therapeutic agent(s) 108 applied to the section 1004 or to a sheath 402 secured to section 1004, similar to the device 900 as described in FIGS. 9A-9D. Therefore, in combination with the delivery through orifice 1020, an additional or secondary delivery of one or more therapeutic agent(s) 108 might be provided as the device 1000 is left in place in the vaginal cavity before, during or after the primary delivery through orifice 1020. The primary and secondary therapeutic agent(s) 108 can be delivered concurrently or, alternatively, sequentially. For example, the agent delivered through orifice 1020 might be fast acting while any agent from the sheath 402 or section 1004 might be slower acting. Still further, the device might be inserted for a time to allow an agent to act from section 1004 before the targeted agent is delivered through the orifice 1020. The various therapeutic agents or API's might be compatible or synergistic when metabolized and introduced to the vaginal cavity in different sequences. In that way, the therapeutic agent(s) 108 can be targeted from the device 1000 and also released as discussed and shown in FIGS. 9A-9D. The vaginal device can be introduced similarly to device 900 and an elongated applicator 920 (see FIG. 9A) may be used to push the device 1000 into the desired position and then disengaged to leave vaginal device 1000 in place. Because of the tube 1010, the applicator may need to accommodate its positioning and thus may have a tear away construction that can be torn away and removed after the device 1000 and tube 1010 are in position, so that therapeutic agent(s) may be introduced from system 1012. After use, the tube 1010 might be used as a removal element to remove the device 1000 from the vaginal cavity.

FIGS. 11A-11D illustrate another embodiment of a vaginal device or system in accordance with features of the invention. The vaginal device 1100 incorporates an insertable vaginal device 1102 that may be inserted into the vaginal cavity. The insertable or insert device can generally resemble the vaginal insert devices 900 or 1700 as described herein in construction, dimension and operation and may be utilized in a similar fashion as those devices. However, vaginal device 1100 also provides the ability to deliver a desired discrete or bolus amount of a therapeutic agent through the insertion process. Specifically, referring to FIG. 11B, the device 1100 contains the insertable vaginal device 1102 inside of an applicator and more specifically an applicator housing 1104. The applicator housing 1104 can be formed as a hollow tubular structure and made from suitable bio-compatible material that is configured and dimensioned to contain the vaginal insert device 1102 proximate to an insertion end 1107. The housing 1104 includes an elongated insertion or push device/structure 1108 that is configured to slide in the housing 1104 and act on the insert device and push or eject the vaginal insert device 1102 for insertion of the device into place in the vaginal canal after first inserting the insertion end 1007 of the housing into the canal. The insertion structure 1108 is configured for the passage of a removal device, such as cord 1110, that is attached to vaginal device 1102 for removal after use, as discussed herein with respect to other embodiments.

In accordance with one feature of the invention, referring to FIG. 11B, the applicator housing 1104 contains the vaginal device 1102 and is configured and dimensioned to also form a dosage zone 1112 positioned forward of the insertable vaginal device 1102 in the housing and between the insertion end and the insert device. As illustrated, the vaginal insert device 1102 includes an elongated body that might have one or more position stability/retention sections 1116 and 1118 that are on either end of the body and may define a longer delivery section 1119 that may or may not be used to further deliver additional therapeutic agents as disclosed herein. In one embodiment, the insert device 1102 resembles device 900 and incorporates the similar elongated body having a delivery section with an outer diameter or cross-sectional diameter that is smaller than the diameter or dimension of the larger position stability section 1116 and the still larger retention section 1118. Once inserted, device 1102 would act in a similar fashion to devices 900 or 1000. The formed dosage zone 1112 is configured to contain, and subsequently eject, a discrete dosage or bolus amount 1114 of one or more suitable therapeutic agent(s). The insertion structure 1108 has an end 1130 that is configured to engage and act on the device 1102 when pushed to eject the device 1102. To that end, the end of the housing 1104 proximate to the dosage zone 1112 may have slits or other features 1134 to allow the device 1102 and dosage amount 1114 to be pushed out of the housing 1104 for application.

Specifically, as illustrated in FIGS. 11C-11D, the housing includes a finger grip portion 1136 that may be engaged simultaneously with the end 1132 of the insertion structure 1108 for pushing the insertion structure into the housing and ejecting the dosage amount 1114 followed by the vaginal device 1102. FIG. 11C illustrates advancement of the insertion structure 1108 and the ejection of the dosage amount 1114 from the end of the housing, such as into a vaginal cavity. FIG. 11C illustrates still further and complete advancement of the insertion structure 1108 and the subsequent ejection of the insertable vaginal device 1102 from the end of the housing, such as into a vaginal cavity behind the dosage amount 1114 of therapeutic agent(s). Once fully inserted, the dosage amount 1114 is delivered into the vaginal canal and the vaginal device 1102 is retained in place and acts to keep the dosage amount contained in the vaginal canal.

In one embodiment, the dosage amount 1114 delivery is the only delivery of the therapeutic agent(s) and the vaginal device 1102 acts only as a physical barrier or plug to contain the dosage amount and prevent leakage while the user is upright and active. In an alternative embodiment, as discussed with respect to devices 900 and 1000, the vaginal device 1102 may also contain a one or more suitable therapeutic agent(s) 108 applied to section 1119 of device 1102 or to a sheath 402 holding one or more therapeutic agent(s) 108 applied to section 1119 of device 1102. In such a scenario, additional therapeutic agent(s) are positioned for release simultaneously and concurrently or sequentially with the dosage amount 1114, similar to the two stage action of the vaginal device 1000 as described herein.

For containing the dosage amount before delivery, the device 1100 might include a removable retention cap 1106 that interfaces with the insertion end 1107 of housing 1104 and covers the housing end and any slits 1130 or other openings that might expose the dosage amount and allow it to leak as shown in FIG. 11A. FIG. 11B shows the cap removed to expose the insertion end 1117. The insertion end 1107 of the housing may be appropriately curved or shaped, as shown in the illustrated embodiment to ensure comfortable insertion of the housing to eject the dosage amount 1114. The cap may be removed prior to insertion of the housing end 1107 and ejection of the contents through pushing the insertion structure 1108 in the housing. Beneath the cap, there will generally be a lubricant or wax or suppository base on the insertion end 1117 that is compatible with the dosed therapeutic agent and exposed when the cap is removed to ensure lubrication and assist in insertion. In one embodiment, the cap can have the lubricant or other material therein when applied.

The dosage amount 1114 of the therapeutic agent may take any form as indicated by the most efficient or effective delivery method such as a solid, like a suppository, or a fluid, a foam, a cream, a gel, or mixtures or combinations of states of matter that may be delivered by pushing the vaginal device 1102 through the dosage zone 1112 of the housing to eject the dosage amount 1114 and insertable vaginal device 1102 therebehind. The housing 1104 and the size of the dosage zone 1112 may be configured and dimensioned and the insertable vaginal device 1102 may be positioned and dimensioned in the housing 1104 to provide for a desired size of the dosage zone 1112 for the proper dosage amount 1114. For example, the insertable vaginal device 1102 might be moved closer or farther from the insertion end of the housing in order to vary the dosage zone 1112 and the contained dosage amount.

In accordance with one feature of the device 1100 as illustrated, a seal or gasket 1020 may be positioned along the outside surface of the body of the vaginal device 1102 at a desired position along the body to seal a portion of the vaginal device against an inside surface of the housing. Referring to FIG. 11B, the seal 1120 is positioned in a groove 1121 in the outside wall of the body of the insertable vaginal device 1102 and sits against the inside wall 1140 of housing 1104. In the illustrated embodiment, the seal is positioned proximate to a positioning element/retention element. The fill zone 1112 can then extend down from the insertion end 1107 of the housing to the seal 1020 for defining dosage zone 1112 and containing the dosage amount 1114. For those therapeutic agent(s) 108 that take a non-solid form such as a fluid, a foam, a cream, a gel, or mixtures or combinations thereof, the seal 1020 ensures that the dosage amount 1114 is contained in the dosage zone 1112 until usage and ejection. Furthermore, the seal can be used to define a more precise volume for the dosage zone 1112 to deliver the desired amounts. As shown in FIG. 11C-11D, as the vaginal device 1102 is pushed out of the insertion end 1107 of the housing 1104, the device 1102 pushes the dosage amount out of the housing and the seal will effectively scrape the internal walls of the housing proximate the dosage zone 1112 for efficient ejection of the dosage amount. The seal is small enough as to not interfere with the comfort and positioning of the device 1102 when it is inserted and positioned in the vaginal cavity.

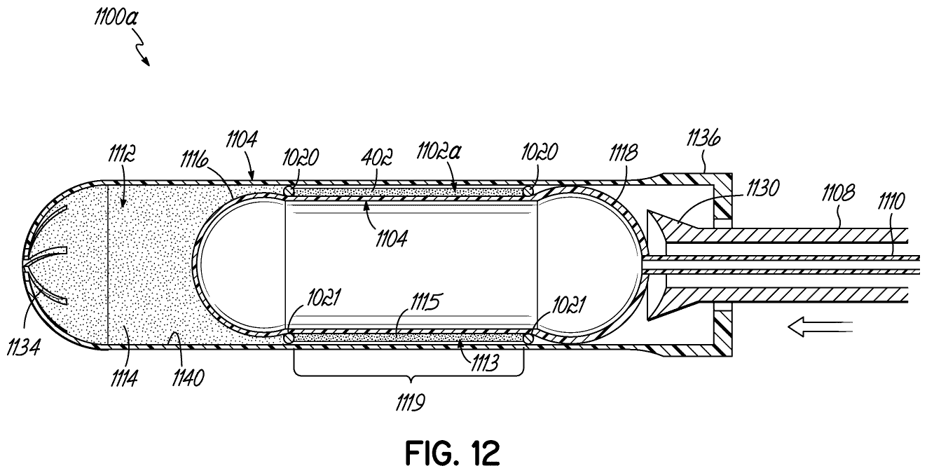

In the embodiment of FIGS. 11A-11D, the device 1100 defines a single dosage zone 1112 that is defined from the seal 1120 proximate section 1116 of the insert device 1102 to the insert end 1107 of the housing 1104. In an alternative embodiment of the invention as illustrated in FIG. 12, one or more additional seals 1020 may be used to define other dosage zones that might be filled and ejected. Specifically, referring to FIG. 12, the device 1100a illustrates an embodiment of the vaginal device that operates generally similarly to vaginal device 1100 in FIGS. 11A-11D so like reference numerals are used as appropriate. The cap 1106 is shown removed in FIG. 12. However, the vaginal device 1100a incorporates at least one additional seal to define other dosage zones to hold other dosage amounts of a therapeutic agent(s) 108. More specifically, the vaginal device 1100a incorporates a seal 1020 positioned toward the other end of the vaginal insert device 1102a, such as proximate a position or retention section 1118. As such, because of the smaller diameter dimension of the section 1119 of the vaginal insert device 1102a, another dosage zone 1113 is formed to contain another dosage amount 1115 of a therapeutic agent(s) 108. That is, dosage zone 1113 is formed between the outer wall of the insertable device 1102a and the housing 1104 and between the seals 1120 at the end sections 1116, 1118. Then, upon activation of the device 1100a as shown in FIGS. 11C-11D, both of the dosage amounts 1114, 1115 are ejected sequentially with the forward seal 1120 and the section 1116 of the body pushing the dosage amount 1114 and the reward seal 1120 pushing the dosage amount 1115 out of the housing and into the vaginal cavity along with the insertable vaginal device 1102a. Each of the seals 1120 will effectively scrape the internal walls of the housing proximate the dosage zone 1112 and the dosage zone 1113 for efficient ejection of the dosage amounts. The seals are small enough as to not interfere with the comfort and positioning of the device 1102a once inserted in the vaginal cavity. Therefore, the device 1100a be used to deliver multiple bolus or discrete amounts of a therapeutic agent 108 from each of the dosage zones 1112, 1113. As may be understood, the dosage amount 1115 may be less than dosage amount 1114 because of size constraints. The post insertion conditions are generally similar to those described with respect to FIG. 11A-11D.

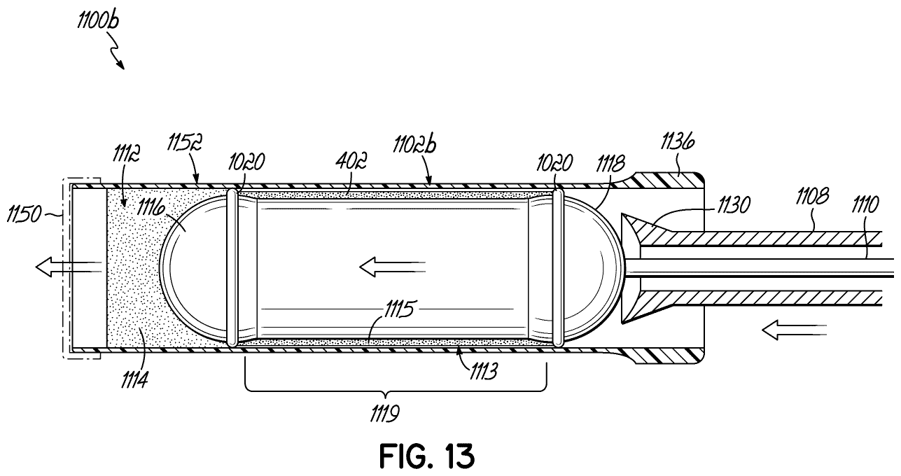

In accordance with one feature of the invention, the vaginal device may use a pre-filled design wherein the particular dosage amount(s) 1114, 1115 are pre-loaded and ready for usage. In accordance with an alternative feature, a selective amount that is determined for a particular dosage, such as by a physician or pharmacist or the user, might be loaded into a housing dosage zone 1112, 1113 and then the housing capped or otherwise sealed for usage. To that end, FIG. 13 illustrates an embodiment of the vaginal device 1100b that operates generally similarly to vaginal devices 1100, 1100a, such that like reference numerals are used as appropriate. The vaginal device 1100b incorporates a removable cap 1150 that seals the end of housing 1152. For particular dosages, a physician or pharmacist might remove the cap 1150 and insert the dosage amount 1114 into the formed dosage zone 1112, and/or might position the dosage amount 1115 into dosage zone 1113, and then the cap might be replaced to seal the housing 1152 until device 1100b is ready for usage. As noted, underneath the cap, a lubricant, wax or suppository base might also be applied with the dosage for lubrication purposes for insertion. In one embodiment, the cap can have the lubricant or other material therein when applied. In another embodiment, the dosage zone 1113, because of its position, might be pre-loaded with the dosage amount 1115, and the selective dosage is just loaded in the front dosage zone 1112 by a physician or pharmacist or the user.

Similar to the vaginal device 1000, the described vaginal devices 1100, 1100a, 1100b can be used to provide a targeted delivery of the therapeutic agent(s) 108 which may be the primary path of delivery through the device 1100, 1100a, 1100b. The dosage amounts 1114 and/or 1115 provide an accurate and complete delivery and the inventive devices 1100, 1100a, 1100b result in more accurate placement and positioning of the therapeutic agent(s) 108. The various dosage amounts 1114, 1115 can be used to target the upper vaginal vascular field and the lower uterine vascular field. Furthermore, the different dosage amounts 1114, 1115 can provide the same sequential and synergistic or multiple agent delivery capabilities as in the device design 1000. As with other embodiments, the insertable vaginal devices 1102, 1102a, 1102b that are inserted and positioned in the vaginal cavity to push in the dosage amounts 1114 and/or 1115, remain in place and act as a physical plug to contain the delivered dosages of therapeutic agent(s) 108 when the user is upright. Such delivery of therapeutic agent(s) 108 may be the primary delivery route and the insertable devices may just act as a barrier to prevent leakage. Alternatively, the devices 1100, 1100a, 1100b may also provide an additional or synergistic delivery of one or more therapeutic agent(s) 108 in conjunction with the primary delivery. To that end, the vaginal devices 1100, 1100a, 1100b might also incorporate a therapeutic agent 108 applied to the body of the insertable device or on a sheath 402 affixed to the body, similar to the devices 900 and 1000 as described herein for an additional or secondary delivery of one or more therapeutic agent(s) 108 in combination with the delivery of the dosage amounts 1114, 1115. The primary and secondary therapeutic agent(s) 108 can be delivered concurrently or alternatively sequentially. For example, the therapeutic agents delivered through the dosage amounts 1114, 1115 might be fast acting while any agent from the insertable device or a sheath 402 positioned on the delivery section he insertable device body might be slower acting. The various therapeutic agents or API's might be compatible or synergistic when metabolized and introduced to the vaginal cavity in different sequences. In that way, the therapeutic agent(s) 108 can be targeted as dosages from the devices 1100, 1100a, 1100b and also released via the respective insertable devices 1102, 1102a, 1102b as discussed and shown with respect to the vaginal device 900 in FIGS. 9A-9D. Once used, the insertable vaginal devices 1102, 1102a may be removed by string 1110 and discarded or reused as desired.

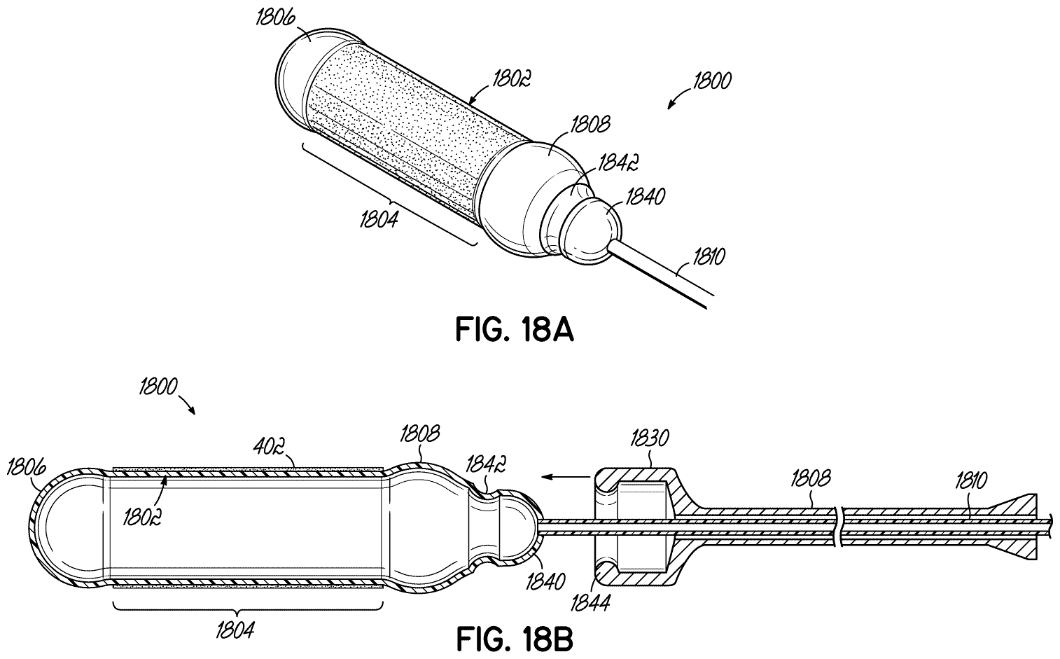

FIGS. 18A-18B illustrate another embodiment of a vaginal device or system in accordance with features of the invention. Vaginal device 1802 may be used in a static sense as discussed herein for device 900 or in a dynamic sense with an application system as discussed with respect to device 1100. Specifically, the vaginal device 1800 incorporates an insertable body 1802 that may be inserted into the vaginal cavity as described herein. The insertable or insert device can generally resemble the vaginal insert devices 900 or 1700 as described herein in construction, dimension and operation and may be utilized in a similar fashion as those devices. FIG. 18A is a perspective view of a vaginal device or insert device 1800 in accordance with one embodiment of the invention and it body 1802 generally tubular or cylindrical in shape. The body 1802 has an elongated form with opposing ends. Portions or sections of the body provide its functionality of holding one or more therapeutic agents 108 and positioning the one or more therapeutic agents 108 for delivery. The body includes a delivery section/portion 1804 between the opposing ends. The vaginal device 1800 includes at least one section that has a larger dimension than the body dimension or the dimension of the delivery section in the form of the position stability or retention sections 1806, 1808, with a diameter dimension that exceeds the diameter dimension of the body delivery section. The larger position stability sections 1806, 1808 may be positioned at the ends of the body and act as retention elements or positioning and stability elements to engage the vaginal wall more firmly than the delivery section and thereby secure and position the device and the delivery section in the proper position in the vaginal cavity for use as disclosed herein.

Vaginal device 1802 incorporates a mating feature for mating with an insertion device 1808. The insertion device 1808 is configured to couple with the insert device and push or eject the vaginal insert device 1800. It might be used to manually position a device for static use. Alternatively, the insertion device 1808 might be used to push a device through an applicator housing as described. Specifically, the vaginal device 1802 includes an engagement feature 1840 that extends from an end of the device, such as from the retention section 1808. The engagement feature is configured to be captured by an end 1830 of the insertion device 1808 that has a complementary shape to the engagement feature 1840. For example, in the illustrated embodiment of FIGS. 18A-18B, the engagement feature 1840 is in the form of a bulb structure having a groove 1842 at its base. The insertion device 1808 has a complementary feature for engaging the engagement feature. For example, the insertion device 1808 incudes a complementary cup end 1830 to receive the bulb structure 1840 therein. A ridge 1844 in the cup end 1830 is configured to snap into the groove 1842 to secure the vaginal device for insertion. The cup end 1830 then can be pulled from the engagement feature 1840 of the vaginal device to free the device once it is inserted. The insertion device 1808 is configured for the passage of a removal device, such as cord 1810, that is attached to vaginal device 1800 for removal after use, as discussed herein with respect to other embodiments.

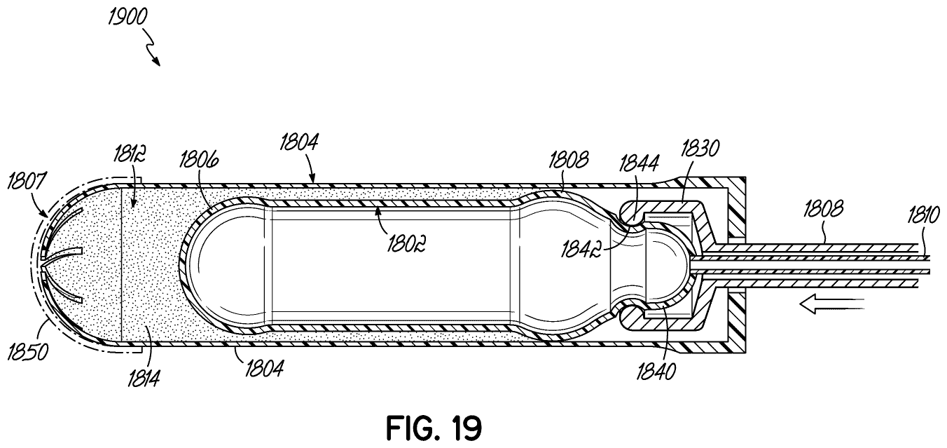

The vaginal device 1800 and insertion device 1808 can also be used in the more dynamic system illustrated in FIGS. 11A-13. That is, it can be used to deliver a desired discrete or bolus amount of a therapeutic agent through the insertion process. Specifically, referring to FIG. 19, the device or system 1900 contains the insertable vaginal device 1802 inside of an applicator and more specifically an applicator housing 1804. The applicator housing 1804 can be formed as a hollow tubular structure and made from suitable bio-compatible material that is configured and dimensioned to contain the vaginal insert device 1802 proximate to an insertion end 1807. The insertion device 1808 is configured to slide in the housing 1804 and act on the device and push or eject the vaginal insert device 1802 for insertion of the device into place in the vaginal canal after first inserting the insertion end 1807 of the housing into the canal. As disclosed herein, the applicator housing 1804 contains the vaginal device 1802 and is configured and dimensioned to also form a dosage zone 1812 positioned forward of the insertable vaginal device 1802 in the housing and between the insertion end and the insert device. As disclosed, the vaginal insert device 1802 includes an elongated body that might have one or more position stability/retention sections. The formed dosage zone 1812 is configured to contain, and subsequently eject, a discrete dosage or bolus amount 1814 of one or more suitable therapeutic agent(s). The vaginal device 1900 may also have a removable cap 1850 that seals the end of housing 1804. As discussed with respect to FIGS. 11A-13, the devices 1800 and 1900 may be loaded and utilized similarly to those other devices 100, 1100a and 1100b as disclosed.

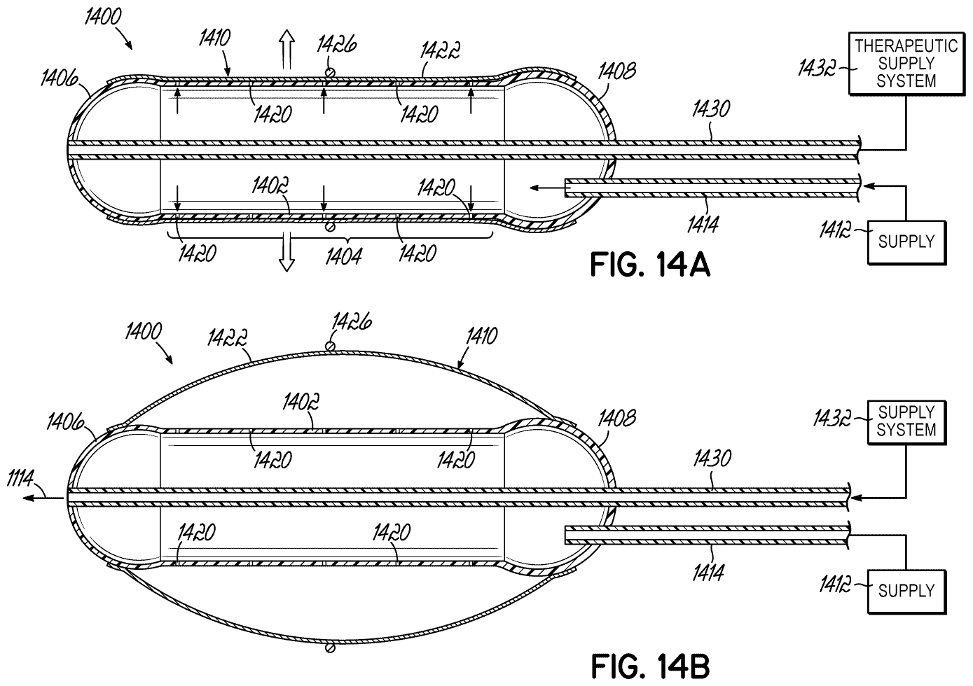

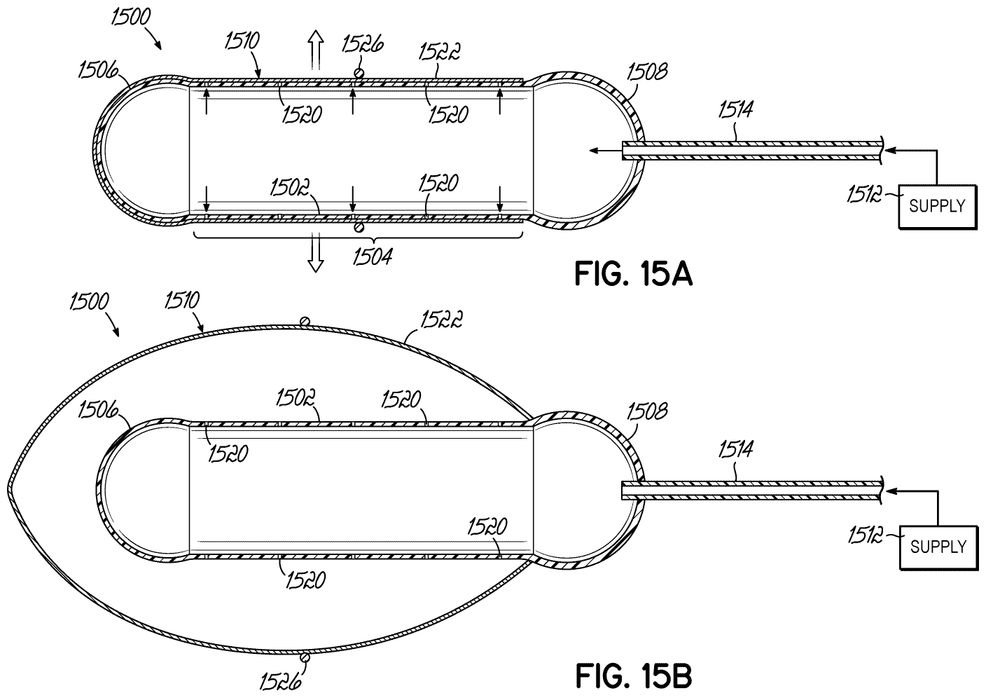

FIGS. 14A-14B and 15A-15B illustrate another dynamic embodiment of the present invention that use shaping components or retention components based on inflation of an element of the vaginal device. The inflatable elements allow for the shape of the vaginal device or specifically the device housing of the vaginal device to be adjusted, molded, shaped, etc. to improve the comfort, fit, function, etc. of the vaginal device. The inflatable elements may be adjusted before, during, or after insertion of the vaginal device into the vaginal canal. In some embodiments, the shaping components may aid in evenly distributing the pressure of a vaginal device against the wall of the vaginal canal aiding in even contact and delivery of a therapeutic agent 108 via a therapeutic sheath. In some embodiments, the therapeutic sheath may stretch or otherwise expand to conform to the new shape of a vaginal device which has been altered via one or more inflatable elements. The inflatable elements of a vaginal device may also act as a retention mechanism which may either hold the vaginal device in a desired location within the vaginal canal or prevent the discharge of fluids, primarily to prevent therapeutic agents 108 from escaping the vaginal canal.

To that end, FIGS. 14A-14B illustrate a vaginal device 1400 that includes a body 1402. The body 1402 might be configured similarly to the vaginal device body of device 900 as described and may be an elongated cylindrical section 1404 having ends 1406 and 1408 for initial retention in the vaginal cavity. Device 1400 includes an inflatable bladder 1410 that surrounds and is affixed to the ends of the device 1400, such as at the position/retention section 1406, 1408. The bladder 1410 is configured and adhered to be airtight around the body 1402. The body 1402 is coupled at one end, such as at the end with retention section 1408, to a supply 1412 of air or gas for inflation through an appropriate inflation tube or capillary 1414. The inflation tube directs air into the interior of the body 1402. The supply 1412 might be a pump or squeezable bladder or other device to deliver pressurized air to the body. The body 1402 includes a plurality of passage holes 1420 that direct a pressurized airflow therein to the outside of the body and into the bladder 1410 to inflate the bladder. The bladder may be inflated to a desired size for fit and application of any therapeutic agents 108 applied to the outside of the bladder. Similar to the delivery zones of the elements discussed herein, such as in device 900, one or more therapeutic agents 108 might be applied onto the outside surface 1422 of the bladder directly or through the use of an adhered sheath, and the expansion of the bladder will direct such therapeutic agents 108 against the walls of the vaginal cavity. If a sheath is used, the sheath would also need to expand with the bladder. As noted, with other embodiments, different sections of the bladder outer surface 1422 may contain different therapeutic agents 108. For further stability, the device 1400 might also incorporate one or more seal or stability rings 1426 thereon for proper retention and positioning in the vaginal cavity. FIG. 14A illustrates the device 1400 pre-inflation and FIG. 14B illustrates the post-inflation state. The larger inflated size enable engagement with a wider diameter or dimension of the vaginal cavity and more tissue contact area than other of the disclosed designs, if that is necessary or desired. The supply 1412 will have a suitable release mechanism for deflating the device 1400 when desired for removal from the vaginal cavity.

In accordance with other features of device 1400, the body 1402 may be configured similar in FIGS. 10A-10B. The vaginal device 1400 also includes a delivery tube or capillary structure 1430 that is in communication with both the device 1400 and a supply and delivery system 1432 of therapeutic agent(s). That supply and delivery system 1432 may include one or more reservoirs or supplies of the therapeutic agent(s) and may also include a mechanized or manual device that couples with the reservoir to deliver the therapeutic agent(s) to the delivery tube as shown and described with respect to device 1000. For example, the system 1432 might include a pipette, a pump, a syringe, or may take the form of a flexible package or reservoir that might be manually squeezed by the user to deliver therapeutic agent(s) from a reservoir to the vaginal device 1400 for targeted usage as disclosed herein. In that way, the device 1400 may be used to deliver multiple therapeutic agent(s) in different vehicles of delivery as discussed herein.

FIGS. 15A-15B illustrate a vaginal device 1500 that includes a body 1502. The body 1502 might be configured similarly to the vaginal device body of device 900 as described and may be an elongated cylindrical section 1504 having ends 1506 and 1508 for initial retention in the vaginal cavity. Device 1500 includes an inflatable bladder 1510 that surrounds and is affixed to at least one of the ends of the device 1500, such as at the retention section 1508, for example. The bladder 1510 is configured and adhered to be airtight around the body 1502. The body 1502 is coupled at one end, such as at the end with retention section 1508, to a supply 1512 of air or gas for inflation through an appropriate inflation tube or capillary 1514. The inflation tube directs air into the interior of the body 1502. The supply 1512 might be a pump or squeezable bladder or other device to deliver pressurized air to the body. The body 1502 includes a plurality of passage holes 1520 that direct a pressurized airflow therein to the outside of the body and into the bladder 1510 to inflate the bladder. The bladder may be inflated to a desired size for fit and application of any therapeutic agents 108 applied to the outside of the bladder. Similar to the delivery zones of the elements discussed herein, such as in device 900, one or more therapeutic agents 108 might be applied onto the outside surface 1522 of the bladder directly or through the use of an adhered sheath, and the expansion of the bladder will direct such therapeutic agents 108 against the walls of the vaginal cavity. If a sheath is used, the sheath would also need to expand with the bladder. As noted, with other embodiments, different sections of the bladder outer surface 1522 may contain different therapeutic agents 108. For further stability, the device 1500 might also incorporate one or more seal or stability rings 1526 thereon for proper retention and positioning in the vaginal cavity. FIG. 15A illustrates the device 1500 pre-inflation and FIG. 15B illustrates the post-inflation state. In the embodiment of device 1500 the bladder covers the end 1506 of the body and inflates over that end. The larger inflated size enable engagement with a wider diameter or dimension of the vaginal cavity and more tissue contact area than other of the disclosed designs, if that is necessary or desired. The supply 1512 will have a suitable release mechanism for deflating the device 1500 when desired for removal from the vaginal cavity.

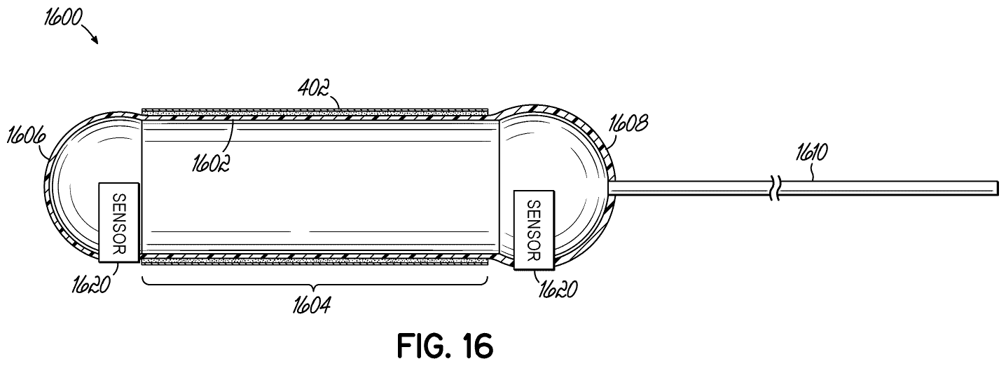

In accordance with another embodiment of the invention, one or more of the devices as illustrated might incorporate one or more sensors as discussed with respect to FIG. 8. FIG. 16 illustrates a device similar to the insertable devices illustrated herein that have a generally tubular body 1602 that has portions or sections that provide its functionality of holding one or more therapeutic agents 108 and positioning the one or more therapeutic agents 108 for delivery. For example, section 1602 might be used for delivering one or more therapeutic agents 108 while end sections 1606, 1608 might be used for positioning and retaining the device in the desired location during use and delivery. One or more sensors 1620 might be implemented in the body, such as at the ends as shown. The positioning of the sensors is not limited to those positions illustrated and they might be positioned elsewhere in body 1602.

Each of the vaginal devices as described herein provide an accurate and complete dosage delivery of a therapeutic agent 108 as described herein. The vaginal devices provide accurate positioning and location and specifically can be positioned for accurate delivery of a therapeutic agent 108 in specific sections and vascular fields of the vaginal area, including the vaginal, uterine, ovarian and peritoneal areas. The construction and length of the vaginal devices and their unique positioning and retention qualities ensure distribution across the specific targeted or required mucosal tissue areas and ensure consistent and continuous tissue contact for delivery of one or more therapeutic agents 108. The devices and features thereof provide retention in the vaginal cavity and canal for complete delivery, assimilation and metabolization of a therapeutic agent 108. Furthermore, the inventive vaginal devices provide the ability to deliver multiple therapeutics/APIs/agents/reagents concurrently or sequentially.

The devices are configured for easy insertion and removal. Furthermore, the various features, as described herein provide for proper positioning, retention and stability of the device in the desired area for delivery of one or more therapeutic agents 108. The devices can be positioned in the lower ⅓ of the vaginal canal to address delivery to the vaginal vascular field or peritoneal vascular field. Alternatively, the devices might be positioned in the upper ⅔ of the vaginal canal to address delivery to the uterine field or ovartian vascular fields. The devices are not limited to their positioning and so might be used for treating both such areas of the vaginal canal. To that end, the shape and geometry of the vaginal devices described herein control the delivery of the therapeutic agent(s) 108 to defined assimilation zones and also maximizes the mucosal tissue contact area for therapeutic assimilation.

While the present invention has been illustrated by the description of the embodiments thereof, and while the embodiments have been described in considerable detail, it is not the intention of the applicant to restrict or in any way limit the scope of the appended claims to such detail. Additional advantages and modifications will readily appear to those skilled in the art. Therefore, the invention in its broader aspects is not limited to the specific details of the representative apparatuses and methods, and illustrative examples shown and described. Accordingly, departures may be made from such details without departure from the spirit or scope of applicant's general inventive concept. Also, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A vaginal delivery system for delivering at least one therapeutic agent comprising:
an insert device including a tubular body having opposing ends;
the body including a delivery section between the opposing ends and having a first dimension;
the body further including at least one retention section having a larger second dimension to act as a retention element for securing the insert device in the vaginal cavity;
at least one therapeutic agent positioned on the delivery section of the body;
the delivery section configured for contacting a vaginal surface of the vaginal cavity when the insert device is inserted into the vaginal cavity to deliver a therapeutic agent thereto.

2. The vaginal delivery system of claim 1 further comprising at least one additional section on the body spaced from the retention section and having a third dimension larger than the first dimension of the delivery section for acting as a positioning element for the body in the vaginal cavity.

3. The vaginal delivery system of claim 2 wherein the retention element and positioning element are positioned at opposing ends of the body.

4. The vaginal delivery system of claim 2 wherein the second dimension of the retention element is larger than the third dimension of the positioning element.

5. The vaginal delivery system of claim 1 wherein the body of the insert device is made of a non-absorbent material.

6. The vaginal delivery system of claim 1 wherein the least one therapeutic agent is applied directly on the delivery section of the body.

7. The vaginal delivery system of claim 1 wherein the least one therapeutic agent is applied on a sheath that is positioned on the delivery section of the body.

8. The vaginal delivery system of claim 1 further comprising an elongated insertion device, the insertion device configured for engaging an end of the insert device for pushing the insert device into position in a vaginal cavity.

9. The vaginal delivery system of claim 8 wherein the insert device includes an engagement feature that extends from an end of the device, the insertion device including a complementary feature for engaging the engagement feature for pushing the insert device into position in a vaginal cavity.

10. The vaginal delivery system of claim 1 further comprising a grip portion positioned at one end of the insert device, the grip portion being configured for being gripped by a user for pushing the insert device into position in a vaginal cavity.

11. A vaginal delivery system for delivering at least one therapeutic agent in a vaginal cavity comprising:
an insert device including a tubular body with opposing ends;
the body having a first dimension;
the body further including at least one retention section having a larger second dimension to act as a retention element for securing the insert device in the vaginal cavity;
an applicator having an insertion end, the applicator configured for containing the insert device and forming a dosage zone between the insertion end and the insert device, the dosage zone configured for containing at least one therapeutic agent;
an insertion device movable in the applicator, the insertion device having an end configured for engaging the insert device and acting on the insert device to eject the insert device and the at least one therapeutic agent from the applicator and into a vaginal cavity.

12. The vaginal delivery system of claim 11 further comprising a dosage amount of the at least one therapeutic agent, the dosage amount contained in the dosage zone for being ejected with the insert device into the vaginal cavity.

13. The vaginal delivery system of claim 11 wherein the insert device body includes a delivery section between the opposing ends and further comprising:
at least one additional therapeutic agent positioned on the delivery section of the body;
the delivery section configured for contacting a vaginal surface of the vaginal cavity when the insert device is inserted to deliver the at least one additional therapeutic agent thereto.

14. The vaginal delivery system of claim 11 further comprising at least one additional section on the body spaced from the retention section and having a third dimension larger than the first dimension of the body for acting as a positioning element for the body in the vaginal cavity.

15. The vaginal delivery system of claim 11 further comprising a seal positioned around the body of the insert device proximate an end of the device and interfacing with an inner wall of the applicator, the seal configured for assisting in the ejection of at least one therapeutic agent from the dosage zone.

16. The vaginal delivery system of claim 15 further comprising an additional seal positioned around the body of the insert device proximate another end of the device and interfacing with an inner wall of the applicator, the seal configured for assisting in the ejection of at least one therapeutic agent from the dosage zone.

17. The vaginal delivery system of claim 16 wherein the seals form an additional dosage zone therebetween that is configured for containing at least one additional therapeutic agent, the insertion device acting on the insert device and seals to eject both the at least one therapeutic agent and the at least one additional therapeutic agent together from the applicator when the insert device is ejected into a vaginal cavity.

18. The vaginal delivery system of claim 15 wherein the seal is positioned proximate to the retention element.

19. The vaginal delivery system of claim 11 wherein the insert device includes an engagement feature that extends from an end of the device, the insertion device including a complementary feature for engaging the engagement feature for pushing the insert device into position in a vaginal cavity.

20. The vaginal delivery system of claim 14 wherein the second dimension of the retention element is larger than the third dimension of the positioning element.
